(19) United States
(12) Patent Application Publication
(10) Pub. No.: US 2010/0297772 A1
(43) Pub. Date: Nov. 25, 2010

(54) METHOD FOR THE CREATION OF GENETIC DIVERSITY IN VIVO

(75) Inventors: Christoph Block, Munster (DE); Claudia Arntz, Lengerich (DE)

Correspondence Address: HENRY M FEIEREISEN, LLC;HENRY M FEIEREISEN, 708 THIRD AVENUE, SUITE 1501, NEW YORK, NY, 10017, US

(73) Assignee: SIGNALOMICS GMBH, Steinfurt (DE)

(21) Appl. No.: 12/090,986

(22) PCT Filed: Oct. 16, 2006
(86) PCT No.: PCT/EP2006/009965

§ 371 Date: Aug. 11, 2010

(30) Foreign Application Priority Data
Oct. 19, 2005 (EP) 05022789.1

(51) Int. Cl. C12N 15/74

(52) U.S. Cl. 435/471

(57) ABSTRACT

Disclosed is a method for creating genetic diversity in vivo, comprising the following steps: fragments of at least one wild-type gene sequence are produced, each of said fragments being provided with at least one region which is homologous with a vector that is suitable for in vivo recombination; randomized bridging oligonucleotides of at least one defined oligonucleotide sequence are produced, parts of the bridging oligonucleotide being homologous with at least one fragment of the wild-type gene sequence; and the linearized vector, at least one wild-type fragment, and the randomized bridging oligonucleotide/s are introduced into an in vivo system for homologous recombination.

The invention relates to a simple and effective method of generating genetic diversity in vivo, in particular of generating the representative DNA libraries, and claims priority to the European patent application 05 022 789.1, whose contents are incorporated by reference.

Generating genetic mutants as the starting material for finding novel proteins with improved properties is of current importance. Nonrecombinative and recombinative methods of mutagenesis are known for this purpose. While, for example, the error-prone PCR (epPCR) and saturation mutagenesis (SM) belong to the nonrecombinative methods, all shuffling approaches belong to the recombinative methods. However, even the shuffling approaches frequently resort to PCR methods for generating the diversity.

Muhlrad et al. (1992) have mutagenized for example the gene for the factor a of the mating pheromone (MFA2) from yeast by means of an epPCR. These results illustrate the disadvantages of the epPCR. This is because the epPCR as such is either based on the insertion of an incorrect base and/or on the absence of the proofreading ability of the polymerase. The inherent property of the polymerase used, however, means that some errors occur more frequently than others. This means that some mutations (such as, for example, transitions) are present in greater frequency than others, and the library is nonrandom (“biased”). This leads to what is known as an error bias and, accordingly, to a lack of representativity on the part of the generated DNA library. Muhlrad et al. (1992), for example, contrast 32 transitions of which 31 are NT to G/C with only 7 transversions, among which G/C to C/G has not been observed at all.

Another phenomenon that is observed in PCR-based methods is what is known as the codon bias, which is based on the nature of the genetic code. Simple point mutations lead to a bias on amino acids which the mutated codon encodes. For example, a point mutation in a valin codon can only lead to six other amino acids, which are Phe, Leu, Iie, Ala, Asp or Gly. In contrast, coding the other amino acids requires either two (C, S, P, H, R, N, T, M, E, Y) or even three point mutations (Q, W, K).

Another disadvantage of the epPCR is that not all bases are accessible for a mutagenization and that, statistically speaking, a given amino acid is only mutagenized into approximately five other amino acids.

A further bias which occurs in any PCR is the amplification bias. A molecule which has been copied early in the amplification process is overrepresented in the final library, or the PCR mix. This may lead to a certain mutant, in contrast, being represented in such small numbers that it is barely detectable in a screening. This problem can be overcome only to some extent by a combination of different (ep) PCRs which are carried out separately and/or by reducing the number of PCR cycles.

Other methods of mutagenesis too, for example what is known as the sequence saturation mutagenesis or the RID method (“random insertion and deletion”), employ PCR in order to generate genetic diversity. This means that the libraries generated by these methods, too, will suffer from a bias—i.e. lack of randomness or representativity—between the variants generated. Moreover, these methods are complicated and laborious.

In contrast to the abovementioned methods in which a longer DNA sequence is mutagenized only in part and randomly, the saturation mutagenesis (SM) generates variants of the DNA sequence in which each amino acid is replaced by all other naturally occurring amino acids. In this method, the number of variants generated will naturally depend on the length of the sequence employed. A very large number of variants result, and all must be tested for their characteristics, such as, for example, for their sequence.

The use of degenerate oligonucleotides is a suitable method for substituting an amino acid by any other or for introducing other directed mutations, and this method has been varied in many ways and developed further. Here, it is possible to incorporate, into the coding sequence, one (or more) synthetic DNA sequences (oligonucleotides/inserts), which may have been mutagenized to a different degree. However, the vast majority of these methods likewise relies on PCR technology, at least for amplifying parts of the genetic variants which have been generated, and therefore suffers from the above-described disadvantages.

In what is known as the gene assembly, a gene is assembled from a plurality of smaller units (“oligonucleotides”). This method can knowingly be exploited for generating diversity by substituting a degenerate oligonucleotide for a wild-type oligonucleotide. In this manner, a mutation is incorporated in the resulting gene. Such a method is known from Stemmer et al. (1994). Again, however, the gene is amplified by means of PCR, and therefore suffers from amplification bias.

Shuffling, which is also known, belongs to the recombinative methods of evolutive mutagenesis. Here, fragments which may, for example, take the form of restriction fragments, are reassembled. Since they take the form of homologous fragments of differing origins (different alleles, different isoforms of an enzyme, different point mutations and the like), this method gives rise to novel genetic variants with modified (if possible improved) characteristics.

In DNA shuffling, the sequence is question is digested with DNasel, the fragments are assembled in a primerless PCR, and the product of this reaction is amplified by means of a PCR. Other shuffling variants, too, employ a PCR, and therefore again suffer from the above-described disadvantages, in particular the amplification bias. If all possible mutants are still to be recorded, however, the number of screened transformants must be increased markedly. This, however, is virtually impossible in practice.

A successful mutagenesis, however, not only requires the generation of representative mutants (a “balanced” and “random” diversity), but also the subsequent transformation of a host organism in order to express the encoded proteins. Here, most of the known methods resort to ligation (for example restriction) and cloning, into a vector, of the library fragments obtained during mutagenesis.

However, this route is generally time-consuming. The diversity of the resulting and expressed DNA library depends on an efficient ligation and bacterial transformation. However, both operations are steps which are error-prone and may lead to a preselection of the mutagenesis products. A low efficacy of one of the two steps results in too small a size of the library. Accordingly, the resulting library is not representative since it does not comprise the required amount of independent clones. This is of importance in particular when more than one position is randomized (mutagenized). In the case of two mutagenized codons, for example, approximately 3100 clones must be screened, in the case of three codons as many as 10⁵ clones, in order to find the least represented double or triple mutant with a probability of 95% (Hogrefe et al. 2002).

Palfrey et al. (2000) describe the effect of the cloning process on the distribution of the mutants in a library. They demonstrate clearly that the distribution of the oligonucleotide sequences which are ligated into the vector pUC19 cannot be recovered in the distribution of the plasmid sequences after cloning. It appears that this effect is not based on a selective pressure since different sequences are favored in differing libraries.

Moreover, plasmid DNA must be extracted for employing the library in other organisms. This means growing the bacteria which harbor the library.

This process, which is probably due to the possible different growth characteristics of the individual clones may mean the loss of the library's complexity.

Bosley and Ostermeier (2005) have developed mathematical approaches for the construction, description and evaluation of libraries. They point out that, following subcloning and/or retransformation, the number of transformants must be much bigger than the number of transformants in the original library in order to maintain the number of independent clones of the original library.

It is known that some of the abovementioned problems can be avoided by using homologous recombination. This is known as the gap repair mechanism from organisms including yeast. Here, free DNA ends undergo recombination with the homologous region in a yeast chromosome. The product is circular and contains the sequence of the chromosome between the recombinogenic ends. Different groups of workers have employed this homologous recombination for the construction of plasmids and the in-vivo cloning. Here, a cleaved vector and an insert whose ends show homology with the ends of the vector are cotransformed into the yeast cells.

It is known to employ homologous recombination not only for the cloning of plasmids in vivo (see above), but also for generating libraries in vivo. Thus, Hua et al. (1998) have generated a two-hybrid cDNA library of human EST clones in a Gal4 AD vector by means of homologous recombination. Fusco et al. (1999), too, have cloned a cDNA library in vivo. Schaerer-Brodbeck and Barberis (2004) have generated a library in yeast by replacing the CDR3 (complementarity determining region) of a variable domain of the light chain of an antibody by homologous recombination in yeast. However, the insert was obtained via PCR, with the disadvantage of amplification bias (see above). The same ultimately also applies to other methods in which in each case either a PCR and/or an epPCR is employed (for example Muhlrad et al. 1992).

In conclusion, it can be said that, in all known mutagenesis methods, the generation and/or the amplification of the library fragments by PCR is limiting with regard to the diversity and representativity of the library. To this are frequently added the disadvantages of cloning involving the steps of ligation and transformation, which also have an adverse effect on characteristics of the library. Moreover, many methods are complicated and/or time consuming and/or expensive.

It is therefore an object of the invention to provide a library which permits a maximum of representative diversity in the selected ranges and can simultaneously be generated as rapidly and simple as possible. The term “representativity” is used in the present context to describe the highest possible balance (lack of bias) and a high randomness of the variants.

The invention achieves this object by the (co)transformation of a host organism, capable of homologous recombination, with wild-type fragments of a selected gene, randomized oligonucleotides (“randomized bridging oligonucleotides”) and a vector. In order to make possible the recombination between these DNA segments, the ends of the wild-type fragments are provided in each case with a region which is homologous to certain vector segments. Equally, the wild-type fragments have a region which is homologous to the randomized bridging oligonucleotide.

For the purposes of the invention, randomized means that bases of an oligonucleotide in a population of oligonucleotides are substituted. In this context, an oligonucleotide which belongs to this population may have only one, or else a plurality of, base substitutions (for example within a codon). Preferably, each oligonucleotide has at least one base substitution in comparison with the wild-type sequence, and each base within the population is substituted by all feasible bases. Accordingly, a “randomized (bridging) oligonucleotide” means, for the purposes of the invention, if at all possible the total of all theoretically feasible oligonucleotide variants of the wild-type sequence based on the position(s) to be randomized. If all feasible variants were covered, the randomization of the mutations would be complete, or “saturated”.

The substitution may also comprise normatural bases or modified bases. The oligonucleotides may comprise one or else more codons. The term “randomized insert” is used synonymously with “randomized oligonucleotide”.

The term “bridging oligonucleotide” was chosen in order to clarify that the oligonucleotide is a bridging member between the fragmented wild-type sequences.

This procedure results in a gene assembly in the host organism while simultaneously incorporating a mutagenized segment, which is the randomized oligonucleotide. The use of homologous recombination allows the cloning steps, which are disadvantageous for the purposes of the invention, to be dispensed with.

In accordance with the invention, only the randomized bridging oligonucleotide ensures the diversity of the DNA library generated. Thus, this region is the only segment which deviates from the wild-type sequence. In order to ensure the representativeness of the library, however, the randomization/mutagenesis is not generated via a PCR, but via non-PCR-based methods. Likewise, an ampification of these fragments by PCR is dispensed with in accordance with the invention. Moreover, the mutagenesis products are transformed into the target organism without intermediate cloning. Since homologous recombination is not mutagenic, the complexity and diversity of the library are retained.

The randomized bridging oligonucleotides are preferably prepared synthetically. The wild-type fragments too may be generated synthetically.

In contrast, it is quite possible to generate the wild-type fragments by means of a PCR based method. Since, however, no diversity is intended to be generated with the fragmenting of the wild-type sequence, no degenerate primers are used so that the use of a PCR for these fragments does not entail the risk of the generated DNA library being unbalanced. All of the overlap regions also lack regions which are mutagenized with regard to the vector sequence, or else to the randomized inserts.

In order to limit the library generated to a size which is manageable in practice, it is advantageous only to randomize one or few codons of an oligonucleotide. The selected oligonucleotides should additionally be as short as possible. However, this randomization should also be “saturated”, i.e. as complete as possible, so that, in total, an acceptable ratio is ensured between library size and the complexity of the screening, which is to follow.

The homologous recombination may take place in host organisms which have a functional recombination system. Organisms which are suitable are not only yeast (S. cerevisiae, other yeasts such as, for example, Schizosaccharomyces pombe), but also bacteria (for example Bacillus subtilis, E. coli), protozoans (plasmodium, toxoplasma), filamentous fungi (for example Ashbya gossypil), plants, or animal cells (for example mammalian cells or chicken DT40 cells).

The homologous recombination in the host organism (for example in yeast, where transformation rates of 2×10⁴ to 3×10⁶ transformants/μg DNA may be achieved) can be influenced by a variety of factors such as, mainly, the length, the design and the number of recombinogenic ends and the molar ratio of vector DNA to insert DNA (wild-type fragments and DNA of the bridging oligonucleotide) which is employed for the transformation. These parameters may also influence each other.

Thus, for example, it is possible for up to 6 fragments to be successfully cotransformed together with the cleaved vector. To this end, for example 10 ng of each wild-type (PCR) fragment and, for example, 200 ng of the linearized vector may be employed. These figures have proven to be possible and meaningful especially in the transformation of yeast.

Equally, it is possible for example to use 100 ng of vector DNA with a 40×molar excess of the insert or else 200 ng of vector DNA with a 5×molar excess of insert DNA. However, it is also possible to employ 100 ng of vector with 1 μg of insert DNA or up to 100 ng of vector with 2 μg of insert DNA plus 1 μg of 4 single-stranded oligonucleotides. Overall, it has emerged that the molar ratio between cleaved vector and insert DNA is preferably between 1:1 to 1:50, especially preferably between 1:1 and 1:40. Optimization is indicated for each system.

It has emerged that an overlap of recombinogenic ends of the fragments of at least in each case 40 bp leads to a particularly high percentage of positive clones, namely 90%. Extending the overlap regions to 80 bp at both ends may lead to up to 98% positive colonies. However, an overlap of 60 bp at both ends will already suffice for 95%. Even an overlap region of only 15 bp still generates a sufficient number of positive clones.

In accordance with the invention, the randomized oligonucleotide should therefore be sufficiently long to make possible a recombination with the wild-type fragment(s) and the vector. The overlap regions preferably comprise at least 10, 20, 30, 40, 50 or 60 bp. Accordingly, the length of the oligonucleotides should amount to at least 23, 43, 63, 83, 103 or 123 bp if only one codon is to be degenerated. If the overlap regions on both sides of the randomized oligonucleotide are chosen so that they differ in length, the oligonucleotide may also assume lengths which are between the numbers given.

The wild-type fragments may assume different lengths which are adapted to suit the size of the gene and the position/length of the randomized bridging oligonucleotide(s).

It is advantageous to carry out a complete randomization at selected positions/sequence segments of the wild-type gene sequence to be mutated. In order to select this position or segment, one should know as much as possible about its function. When the amino acid(s) or the region is determined, the primers for generating the wild-type fragments may be selected in such a way that the wild-type fragments encompass the region in question, or, if only one wild-type fragment is used, abut with the region to be mutagenized. In accordance with the invention, the determination of the wild-type fragments should therefore correspond with the location and position of the DNA sequence segment to be mutagenized.

In an advantageous embodiment, the method according to the invention is carried out in yeast as the host organism and is combined with a two-hybrid system or variants or developments thereof for evaluating the interaction between two binding proteins. In one such system, a double selection takes place: only those yeast colonies are capable of growth which have undergone a recombination, since the recombination has taken place in a gene for a binding partner and thus in a gene which is required for selective growth; all fragments must have been incorporated. In this case, therefore, a selection is made not only for the presence of the circular vector, as is the case in traditional systems, but additionally for the presence and functionality of the gene which has undergone recombination. The mutation efficacy in the transformants obtained is, therefore, 100%.

When using a two-hybrid system or a development thereof, it is advantageous that homology regions which are located in the vector and which are used for the homologous recombination are not identical between the two vectors employed, since otherwise recombination between the mutagenized fragment and the second vector in the cell may take place.

In addition, the present invention is rapid, simple and variable since the use of wild-type fragments and randomized oligonucleotides and their subsequent homologous recombination means that restriction cleavage sites need not be present in the gene in each case. The variation of the oligonucleotide sequence, for example via synthetic methods, and of the abutting fragments, for example via PCR-based or else synthetic methods, in contrast, is simple to carry out.

The use of PCR wild-type fragments in combination with one or more randomized oligonucleotides—instead of, for example, the exclusive use of oligonucleotides—increases the flexibility of the method. Thus, it is possible to select, for example in the case of longer genes, restriction cleavage sites in the vector, and it is not necessary to generate silent mutations for linearizing the vector.

If a plurality of mutations are to be performed which are located in different regions of the gene to be mutagenized, it is frequently possible to retain the vector primers. Again, this makes the present method simple and inexpensive.

In the method according to the invention, the randomized oligonucleotides are preferably single-stranded and comprise the desired randomizations/mutations at one or more positions. This advantageously avoids the necessity of having to synthesize the complementary strand by means of a PCR.

In an advantageous development, the randomized single-stranded oligonucleotides may be employed as forward and reverse oligonucleotides. This method leads to an improved transformation efficacy. Although one would expect the formation of a heteroduplex between oligonucleotides which are not fully complementary—and thus an increased formation of undesired double clones, this procedure, surprisingly, does not entail this expected disadvantage. This embodiment therefore entails the advantage of an increased transformation of efficacy without the disadvantage of an increase in the number of double clones.

A particular advantage of the method according to the invention is that the desired DNA library is constructed directly in the target (host) organism and not beforehand in vitro by PCR, which entails the above-described disadvantages. Moreover, the risk of potentiating errors which may happen during the synthesis of these oligonucleotides is limited, as long as only one or few randomized oligonucleotides are used.

Furthermore, it is advantageous that no degenerate primers are employed for the PCR-based amplification of the wild-type fragments. The formation of mutants which are similar to the wild type (primers bind best to a sequence with a maximum of similarity) can therefore be avoided. A similar effect which may occur in homologous recombination (for example) in yeast can be avoided by the fact that the overlap regions between the randomized oligonucleotide and the fragments preferably do not comprise any mutations.

The desired DNA gene library advantageously does not comprise any original wild-type sequence. To avoid as much as possible, or to minimize, such a “contamination” of the desired DNA gene library by original wild-type sequences (template DNA), it is possible to elute the wild-type fragments from the gel after they have been generated and then to employ only these fragments together with the randomized insets) in the transformation.

The figures show examples of the position of the randomized bridging oligonucleotide. It is possible for individual positions or a plurality of positions (codons or individual bases) of the bridging oligonucleotide to be randomized.

### a. PRINCIPLE OF THE METHOD ACCORDING TO THE INVENTION

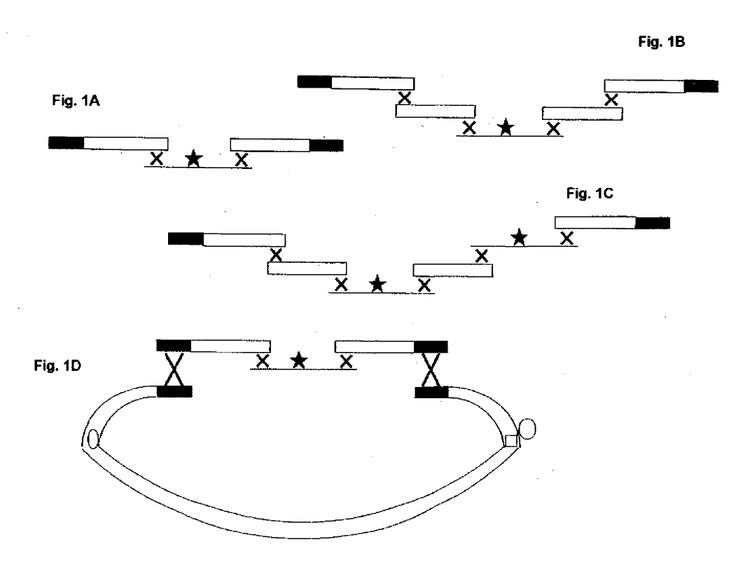

FIG. 1 shows the principle of the method according to the invention. The asterisks symbolize (different) randomized regions of the bridging oligonucleotide, while the gray bars represent vector regions or homologies with vector regions. The light bars represent segments of the gene which is to be mutagenized by the bridging oligonucleotides. These “light” gene segments, however, correspond to the wild-type regions in question, and are therefore not randomized themselves. The crosses symbolize the overlap regions and therefore mark the region of the homologous recombination.

FIGS. 1A and 1B show different embodiments of the invention for introducing the randomized oligonucleotide(s). FIGS. 1A and 1B show the method with only one bridging oligonucleotide, which may comprise for example one or more randomized codons. FIG. 1B shows by way of example the recombination of four wild-type fragments and one randomized oligonucleotide. These may take the form of, for example, three or more wild-type fragments.

Another possibility is the use of only one wild-type fragment which, in the following steps, is used together with one or more randomized bridging oligonucleotides.

FIG. 1C shows the combination of two bridging oligonucleotides with four wild-type fragments. This figure is intended to show, by way of example, that various combinations regarding number and length of the respective fragments/oligonucleotides are possible.

A combination of two randomized bridging oligonucleotides with one another with a different number of wild-type fragments is also feasible.

FIG. 1D shows the integration, into a vector, of the situation in FIG. 1A by homologous recombination.

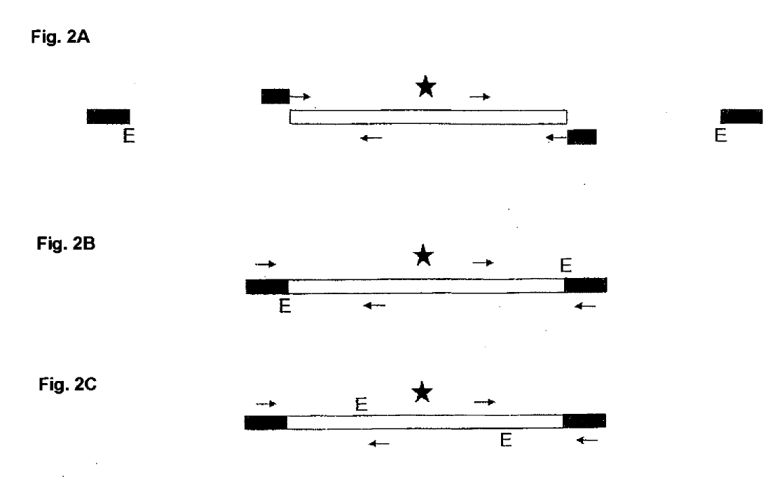

FIG. 2 shows what the structure of the integration vector and the wild-type fragments for the PCR in order to generate the wild-type fragments for the homologous recombination might look like. The stars symbolize the position of (one) randomized codon, the gray bars indicate the vector regions or homologies with vector regions, the light bars indicate the wild-type regions of the gene to be mutagenized, and E indicates the recognition sequence of a restriction enzyme.

FIG. 2A illustrates the case where the gene to be mutagenized is not present in the vector employed in the transformation. Here, the external primers which are employed for synthesizing the PCR fragments may have a region with homology to the vector. The vector may be cleaved with the enzyme E.

The advantage of this embodiment is that the gene to be mutagenized need not be cloned into the vector. The external primers used are advantageously rather long in order to ensure effective recombination.

FIG. 2B shows the situation where the gene to be mutagenized is present in the vector employed in the transformation.

The advantage of this situation is that, as the result of the choice of the primer, the homology region between the vector with wild-type fragments and the mutagenized gene fragment can have any desired size; the primers used need not comprise the homology region for the in-vivo recombination, as has been shown in FIG. 2A.

FIG. 2C shows this situation after the insertion of silent mutations for the generation of restriction cleavage sites (E). The silent mutations may be selected by means of a publicly available computer program (for example http://watcut.uwaterloo.ca/watcut/watcut/template.php). These cleavage sites may be introduced in order to determine effect of a longer homology region between fragments and vector on the efficacy of the method according to the invention.

To avoid wild-type background (“contamination”; see above) in the desired DNA library, what is known as a stuffer fragment (Sneeden and Loeb, 2003) may advantageously be cloned into the restriction cleavage sites E or into other suitable restriction cleavage sites within the gene or the vector; the sequence of this fragment is entirely heterologous to the gene to be mutagenized. If the vector comprising the stuffer fragment is cleaved with the cloned enzymes, the band of the fully digested vector can, firstly, be distinguished readily on the gel from the once-cleaved vector; secondly, the transformation of the vector fragment into the host organism does not give rise to wild-type background as the result of religation, since the vector with the stuffer fragment is not functional.

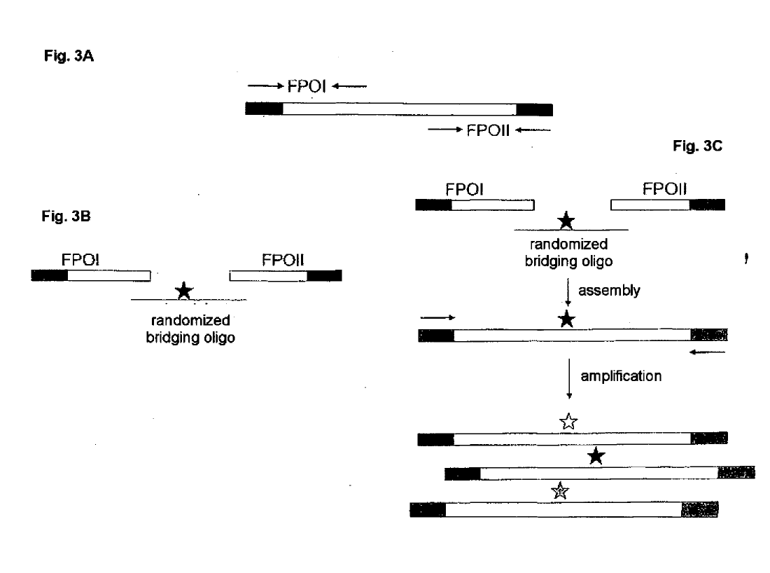

FIG. 3 shows the method according to the invention (FPO means “fragment plus oligonucleotide”). The regions shown in dark are the homology regions between the vector and the wild-type DNA or the randomized bridging oligonucleotide; the stars symbolize the randomized codon. When generating the wild-type fragments, a proofreading polymerase may be employed. This minimizes errors occurring as the result of the incorporation of incorrect bases.

FIG. 3A shows the synthesis of two wild-type fragments. In this method, it is also possible to synthesize one or more than two fragments and to employ these for the subsequent reactions. Likewise, it is possible to employ a plurality of bridging oligos.

Another possibility is the synthesis of only one wild-type fragment which is used in the following steps together with one or more randomized bridging oligonucleotide(s).

The next step is the transformation, into the host organism, of the two wild-type fragments with the bridging oligonucleotide which may carry randomized codons or bases at one or more sites (FIG. 3B).

The method according to the invention may be partly verified in vitro. To this end, an assembly with the oligonucleotide in a primerless PCR may be carried out, as is shown in FIG. 3C. The products of this reaction are then employed as templates for an amplification with the two external primers; this is followed by the transformation of the amplicons together with the cleaved vector into the yeast cells, in which the in-vivo recombination can subsequently take place.

In the method according to the invention, it is neither necessary for the bridging oligonucleotide to bind to template DNA nor do the mismatches have to be located in the homology region. There is therefore a high degree of freedom for the position(s) of the randomization, because they do not affect the homologous recombination in the host organism. By using a plurality of bridging oligonucleotides (see also FIG. 1C), it is possible simultaneously to insert a plurality of mutations even at positions which are further removed.

In contrast to the pure gene assembly, where a gene is assembled from individual oligonucleotides, the present method only requires few oligonucleotides, which makes the method both less expensive and less error-prone, because it is frequently not the PCR reaction which is the limiting factor of these gene assembly methods, but the quality of the oligonucleotides.

In addition, this case simplifies the annealing between the fragments because only few recombinogenic ends for an in-vivo recombination exist.

2. Preliminary Tests for the Method According to the Invention

a. Generation of a Gene Library by Homologous Recombination

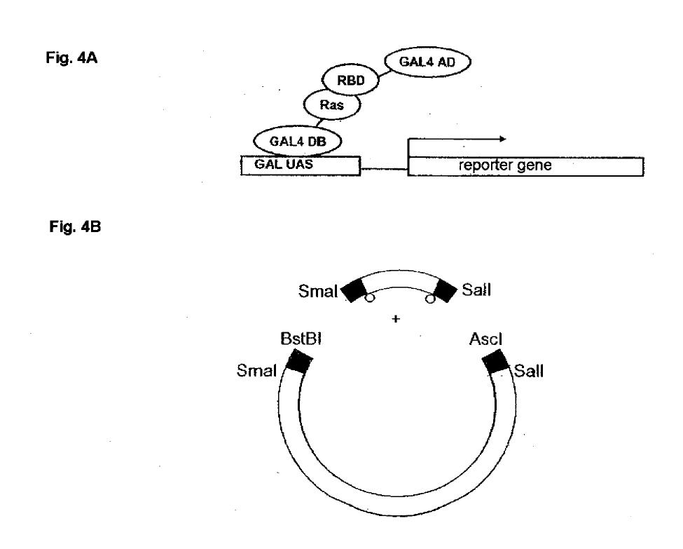

The purpose of this test is, firstly, to prove that the principle of homologous recombination is suitable for generating a DNA library. To this end, a two-hybrid system was used which tests the interaction between Ras and the Ras-binding domain (RafRBD) of the protein kinase Raf enzyme (see FIG. 4A).

In the two preliminary tests described hereinbelow, the fact that the result to be expected is already known is exploited. The homologous recombination in vivo is thus verified in connection with generating a library with a view to functionality.

The known two-hybrid system was carried out with the aid of methods known to the skilled worker.

1. Identification (“Hitpicking”) of the Known RafRBD Mutants

The RafRBD enzyme is known to have different mutants (Block et al., 1996) which differ with regard to their binding affinities, in the following order by increasing binding affinity:

RafRBD−R67A<T68A<V69A<WT (wild type) 1

A mutant A85K is additionally known (Fridman et al., 2000), and the binding affinity of this mutant is above that of the wild type. If these mutants are studied in the known two-hybrid system under the expression conditions described in Jaitner et al. (1997), this range is confirmed, but the gradation between WT and A85K is not reliable.

As proof of the suitability of homologous recombination for generating DNA libraries, silent mutations (BstBI/AscI) were inserted into the WT-RafRBD gene, and a stuffier fragment was cloned in. Thereafter, it was possible to cleave the vector with these enzymes and to use it for transformation purposes (see FIG. 4B).

The above-described RafRBD genes (R57A, T68A, V69A, A85K) and the wild-type gene were excized completely from their vectors with SmaI/SalI (see FIG. 4B) and eluted from the gel.

Vector and inserts were employed for the transformation of yeast in a molar ratio of 1:4; a total of 1 μg DNA was transformed in the process. A molar ratio of 1:6 gave no improvement of the transformation rate, which was 2 to 5×10³ per μg DNA.

TABLE 1
Results of the hitpicking of the known RafRBD mutants. The
fluorophore employed was RedStar.

| | Result of the quantitative screening | | | |
|---|---|---|---|---|
| | picked mutant | | | |
| | A85 = alanine = | | | |
| | wild type | | A85K = lysine | |
| Description of the mixture | Number | % | Number | % |
| All mutant fragments, | 12 | 24.5 | 37 | 75.5 |
| mixed, plus cleaved | | | | |
| vector | | | | |
| Circular vectors of all | 50 | 76.9 | 15 | 23.1 |
| mutants, mixed | | | | |

As can be seen from Table 1—and what was expected for the two-hybrid system—only the wild type and the improved mutant A85K have been detected. This proves the suitability of homologous recombination for the generation of libraries in vivo.

(ii) Identification of Mutants (“Hitpicking”) Using a Fragment with a Randomized Codon.

The randomized codon was introduced at amino acid position 85 of the RafRBD via random mutagenesis, following the method of Zheng et al. (2004) (see in detail under item 6). In the wild type, this position is occupied by an alanine. The mutant A85K (see above), which is markedly improved, and the mutant A85R, whose affinity for Ras is between that of the WT and of A85K, are known from the literature (Fridman et al., 2000). A reliable distinction between these improved mutants and the wild type is not possible in the two-hybrid system (own data, unpublished).

In order to achieve a reliable discrimination between these two mutations, the so-called n-hybrid system was used. This system is a development of the known two-hybrid system which is based on the principle of carrying out the known hybrid system in such as way that the binding reaction between the first ligand (first hybrid, or fusion, protein) and the second ligand (second hybrid, or fusion, protein) is deliberately “made worse” by the choice of suitable reaction conditions. A “worse situation” always exists when the equilibrium (flow equilibrium) of the reaction of the formation of a ligand complex is shifted to favor the ligands (starting materials). The binding reaction between the ligands is therefore inhibited or slowed down. The ligand complex is defined over the ligands by a functional transcription factor.

If, however, one of the ligands of the starting system is replaced for example by a ligand with a markedly higher affinity for the respective other binding partner, the “worst situation” of the system is overcome, and correspondingly more ligand complexes are formed in comparison with the starting situation. The use of a ligand with higher affinity therefore leads to an enhanced expression of the reporter gene—whose activity can be detected quantitatively—in comparison with the interrupted starting situation. This makes possible in particular the presentation of the relative activity of an interaction pair in contrast to a comparative pair.

This n-hybrid system is suitable especially as a screening method for comparing ligands with affinity and with high affinity. To make possible this comparison, for example the reporter gene expression of a wild-type ligand may be used as a reference value (100%). On this basis, the affinities of mutants of this wild type can be shown as relative parameters based on the affinity of the wild type.

In the embodiment of the n-hybrid system which has been chosen here, the competitor was employed to “make worse” the binding reaction. The competitor binds to one of the hybrid proteins and therefore inhibits, or delays, the formation of the ligand complex within the meaning of a competitive or noncompetitive inhibitor.

In accordance with the invention, it is possible to employ a competitor both for the prey protein and for the bait protein. If a competitor were selected for the prey protein—and therefore the formation of the ligand complex made worse a priori—but if the affinity of the prey protein for the bait protein were to exceed the affinity of the competitor employed to the bait protein, then the equilibrium of the ligand binding reaction is, again, influenced in favor of the ligand complex binding. The high-affinity prey protein thus becomes quantitatively detectable as the result of the expression of the reporter gene, which is correspondingly high. This high-affinity reaction would not be detectable if the detection level of the system were already exceeded.

To carry out the preliminary test described here, the wild-type RafRBD without fusion protein on the GAL4 activation domain is employed as competitor. This protein binds to Ras, with which it forms an inactive complex. It competes with the mutants for the binding and thus permits an increased discrimination between improved mutants of RafRBD and wild-type RafRBD.

The principle and the procedure of the n-hybrid system are described in detail under item 4.

The insert which in this case comprises a randomized codon at position 85 of the RafRBD was excized from the library DNA with SmaI/SalI and, after gel elution, transformed into yeast with the BstBI/AscI-cleaved vector (see FIG. 4B).

The vector and the insert were employed in a molar ratio of 1:4 to transform the yeast; a total of 1 μg of DNA was transformed.

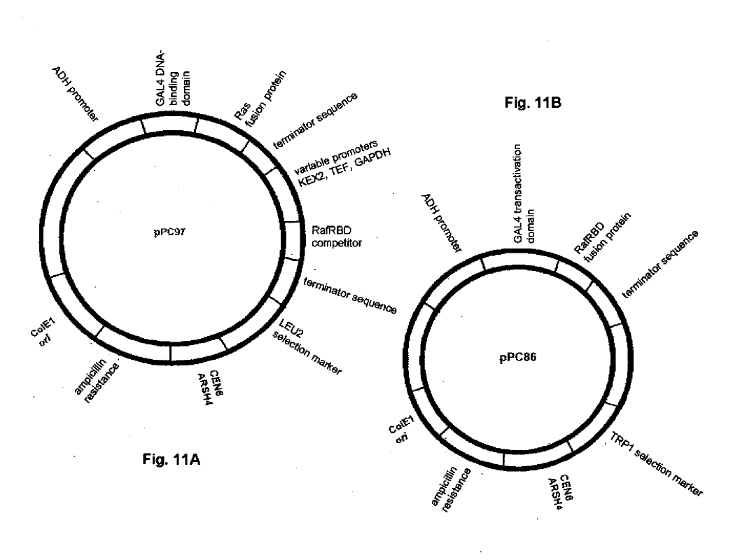

In all approaches, either pPC97-ras (for the two-hybrid) or pPC97-ras with the competitor TEF promoter/RafRBD (for the n-hybrid system, see FIG. 11) is cotransformed as the second plasmid.

Using in-vivo homologous recombination, it was possible to discriminate between the wild type and the improved mutant by employing a quantitative screening with hitpicking the most fluorescent clones (see Table 1).

TABLE 2
Hitpicking result. The results of hitpicking when employing
the two-hybrid and the n-hybrid system are compared. The
fluorophore employed was RedStar.

| | |
|---|---|
| Two-hybrid | n-Hybrid |

| Hit | Number | % | Number | % |
|---|---|---|---|---|
| K(Lys) | 10 | 25 | 18 | 45 |
| R(Arg) | 13 | 32.5 | 19 | 47.5 |
| A(Ala) | 7 | 17.5 | 2 | 5 |
| P(Pro) | 5 | 12.5 | | |
| G(Gly) | 2 | 5 | | |
| S(Ser) | 2 | 5 | | |
| 1 x each | Q | 2.5 | T | 2.5 |

Table 1 shows clearly that homologous recombination was functional in both systems and, as expected (and comparative with the use of a circular vector, author's own data, unpublished), gives the better results in the n-hybrid: when employing the n-hybrid system, the variability is much lower than in the two-hybrid system; the expected mutants A85R and A85K were found at more than 90%.

The negative control consisted in transforming the blank vector; as expected, no colonies were discernible.

The sequences of the clones obtained after homologous recombination were analyzed in greater detail; all showed precise recombination. This was expected since only those clones in which recombination takes place in a base-precise manner are capable of growing on the select media with aminotriazole as the competitive inhibitor for His expression. With the same protein-protein interaction, the reporter gene readout corresponds to the higher selection pressure on the His3 gene. The addition of 50 mM aminotriazole results in the selection of only those host cells which contain fusion protein with sufficiently high affinity. This is because the affinity of the binding partners must be sufficiently high for a sufficient amount of His (or the reporter gene) to be expressed, despite the growth on a competitive His expression inhibitor.

This means that a double selection of the clones takes place in this particularly advantageous embodiment of the n-hybrid system: the system selects for the presence of the vector with its growth marker and, in parallel, for successful recombination of one of the two binding partners.

b) Use of In-Vitro Assembly of the Fragments and of the Randomized Bridging Oligonucleotide Before Transformation into Yeast

This preliminary test is intended to demonstrate the amplification bias. The preliminary test follows the principle shown in FIG. 3C.

For the in-vitro assembly, in each case 20 ng or 40 ng of the synthesized fragments FPOI and FPOII were assembled with a four-fold molar excess of the randomized bridging oligonucleotide in a primerless PCR. The mixture had a volume of 50 μl. The PCR program was as follows:

94° C. 2 min, 30×(94° C. 30 sec, 60° C. 30 sec, 72° C. 60 sec), 72° C. 10 min

In an second experiment, 40 ng of synthesized fragments were employed, and the assembly was carried out at 64.8 and 65.8° C.

To amplify the assembly product, 1 μl of the assembly mixture was employed in a PCR. The primers used were FPO-out-for and FPO-out-rev. The program was the same as for the assembly reaction. In each case 5 PCR reactions were carried out, and combined after the amplification, in order to minimize the occurrence of any prominent PCR products (amplification bias).

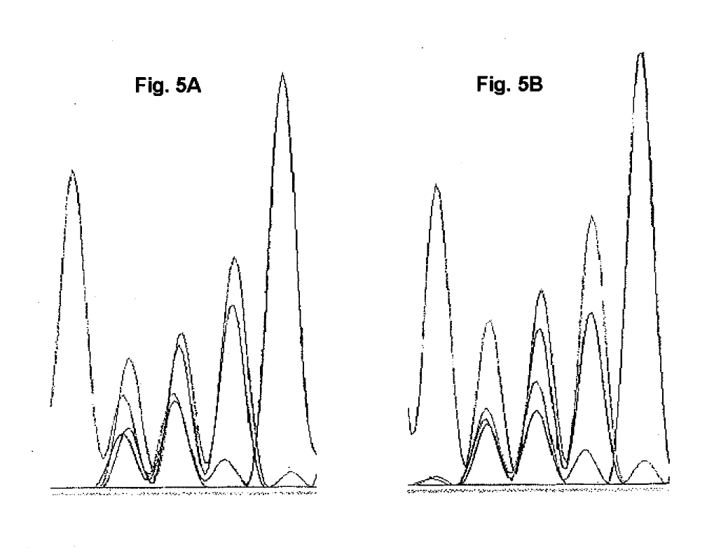

The PCR fragments obtained after the in-vitro assembly were sequenced. FIG. 5A shows the result by way of example. The base distribution is relatively uniform, but not ideal, which is attributed to the occurrence of an amplification bias, even though parallel reactions were carried out.

After the in-vitro assembly, the DNA was restricted with Xma/SaI, ligated into the vector which had been cut with the same enzymes and transformed into bacteria. The DNA (=library DNA) was extracted from the bacteria with Qiagen's Miniprep kit and sequenced. FIG. 5B shows the sequencing processes. It can be seen clearly that one base, in the present case T, is even more overrepresented in comparison with the sequence of the PCR product (see FIG. 5A). This phenomenon, which is probably due to the possible different growth properties of the individual clones, is also described by Schaerer-Brodbeck and Barberis (2004). This process may mean the loss of the library's complexity.

Again, this result shows clearly that homologous recombination in vivo is preferable to cloning, and that an in-vitro assembly also entails a bias of the PCR product.

The library DNA (circular plasmid) and the PCR products after the amplification of the assembly products were also employed for the transformation of yeast (in the case of the fragments, the cleaved vector—for the enzymes used, see Table 3—was added).

In all reactions, pPC97-ras with the competitor TEF promoter/RafRBD (for the n-hybrid system, see FIG. 11) was cotransformed as the second plasmid.

TABLE 3
Comparison of the hitpicking results.

| | | | Fluorophore | | | | | |
|---|---|---|---|---|---|---|---|---|
| | Fluorophore | | cobA | | | | Fluor. RedStar | |
| | cobA | | BstBI/AscI | | Fluor. RedStar | | XmaI/SalI | |
| | Circular | | Assembly plus | | Circular | | Assembly plus | |
| | vector 40 K | | vector 28 K | | vector 57 K | | vector 22 K | |
| | (plus 7 DC) | | (plus 2 DC) | | (plus 3 DC) | | (plus 1 DC) | |
| Hit | Number | % | Number | % | Number | % | Number | % |
| K (Lys) | 13 | 32.5 | 8 | 28.6 | 31 | 54.4 | 10 | 45.5 |
| R (Arg) | 24 | 60.0 | 17 | 60.7 | 23 | 40.4 | 12 | 54.5 |
| A (Ala) | | | | | | | | |
| P (Pro) | | | | | 1 | 1.8 | | |
| G (Gly) | 2 | 5.0 | 3 | 10.7 | 1 | 1.8 | | |
| S (Ser) | 1 | 2.5 | | | 1 | 1.8 | | |
| Others | | | | | | | | |

Assembly plus vector: use of the PCR-amplified in-vitro assembly product for the in-vivo recombination;
circular vector: use of the library DNA (circular plasmid) for the transformation of yeast;
DC = double clone.
The fluorophore used is given in the first line; the restriction enzymes with which the vector, if used, has been cleaved are indicated in the second line. Here, the enzyme combination BstBI/AscI creates an overlap region of fragment and vector of 153/130 bp; in the case of XmaI/SalI this overlap region is 64/61 bp in size.

It can be seen from Table 3 that the use of circular vector and fragment plus vector (FPO) leads to comparable results in the hitpicking method; that is to say that the homologous recombination works just as well as the use of circular plasmid. As expected, the improved mutants are recovered when using the n-hybrid system (cf. Table 2) at around 90%.

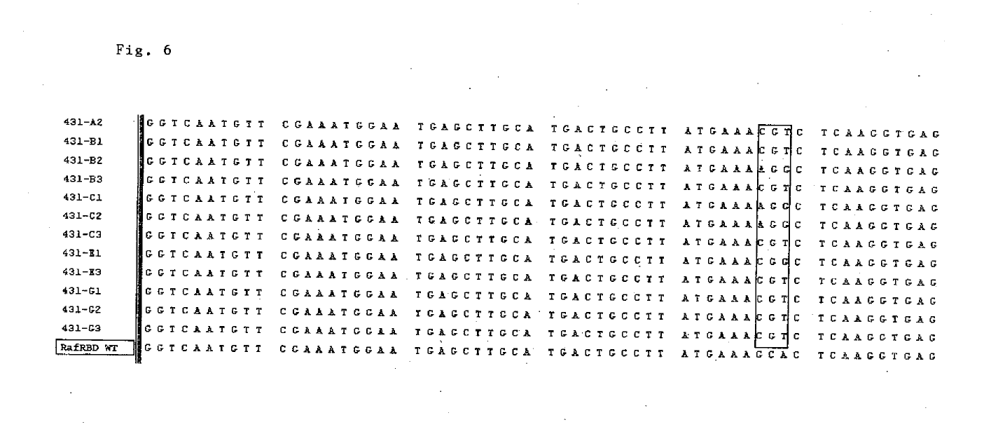

An analysis of the sequences in the event that RedStar is being used and fragment plus vector are employed (Table 3, right column), a codon bias for arginine (R) can be observed. In 8 out of 12 cases (=66.7% 3×AGG, 1×CGG), CGT has been found (see FIG. 6; what is shown is the sequence of part of the RafRBD of 12 clones in comparison with the wild-type sequence (WT=alanine=GCA), the box indicates the randomized position). The expected values would be approximately 33% for each codon.

Double clones are detected in all mixtures. In these clones, sequence analysis demonstrates that two different codons are present at this position. This means that two circular library plasmids have been taken up, or two different fragments have been incorporated into two restricted vectors which have been taken up.

The amplification bias was detected directly when another gene was mutagenized (use of the in-vitro generation of the library by PCR and passage via E. coli, author's own data, unpublished). Glycine was the improved mutant which had been found seventeen times. In this case, only GGG codes for this amino acid, although GGT, too (also use of NNG/T as degenerate codon) codes glycine. An analysis of the complete sequence of all seventeen clones demonstrated that a “wobble” had occurred at another position of the sequence, and this wobble was recovered in all clones. This is only possible when the wobble has occurred in one of the first cycles of the PCR reaction and this template has then been amplified during the course of the subsequent cycles.

Again, this result demonstrates the effects which an amplification bias may have. The result may be that some mutants are present in the library at a very low percentage only, and it is therefore not guaranteed that all mutants which are possible are indeed present in the target organism in statistic proportions—the amount of colonies to be screened is increased.

This effect is particularly drastic in those amino acids which are encoded only by one codon, such as methionine and tryptophan.

If mutants are only rarely present, they are only found rarely when a particular percentage of hits is analyzed. Thus, individual mutants may escape detection if they have been observed only few times in comparison with frequent, also improved mutants.

PCR errors may also be propagated in this manner—these errors are not observed in other cases when the error which has occurred does not take the form of a wobble, as in the present case, but an error which impacts on the reading frame, or is the incorporation of a different amino acid. Since, in such a case, the colonies do not grow, this phenomenon might drastically reduce the transformation efficacy. In the case where a heterologous amino acid is incorporated, this might also falsify the screening result since the undesired mutation might impact on the effect of the intended mutation, which might thus escape detection.

3. Performing of the Method According to the Invention Using Robot-Assisted Hitpickinq

a) The method according to the invention was carried out in a two-hybrid and an n-hybrid system (see in detail also item 40).

Vector Construction

To construct the vector, silent mutations for generating BstBI and AscI site were introduced into the RafRBD gene, which is present in the vector pPC86-RafRBD with kanamycin resistance. To this end, the following primers were employed in a QuikChange Multi reaction:

QC-RafRBD-BstBI
AAGAACAGTGGTCAATGTTCGAAATGGAATGAGCTTG
QC-RafRBD-AscI
ACGAACACAAAGGTAAAAAGGCGCGCCTAGATTGGAATACTGATGCTG

The bases which are modified in comparison with the wild-type sequence are shown in bold.

After verification by restriction sequencing, a 2.6 kb stuffer fragment was cloned into these restriction sites, which fragment had been amplified with a different vector as template, using suitable primers.

This vector with stuffer fragment was then cleaved with BstBI/AscI or Smal(or XmaI)/SalI and, together with the fragments/the randomized bridging oligonucleotide, transformed into yeast for the homologous recombination.

(ii) Generation of the Wild-Type Fragments

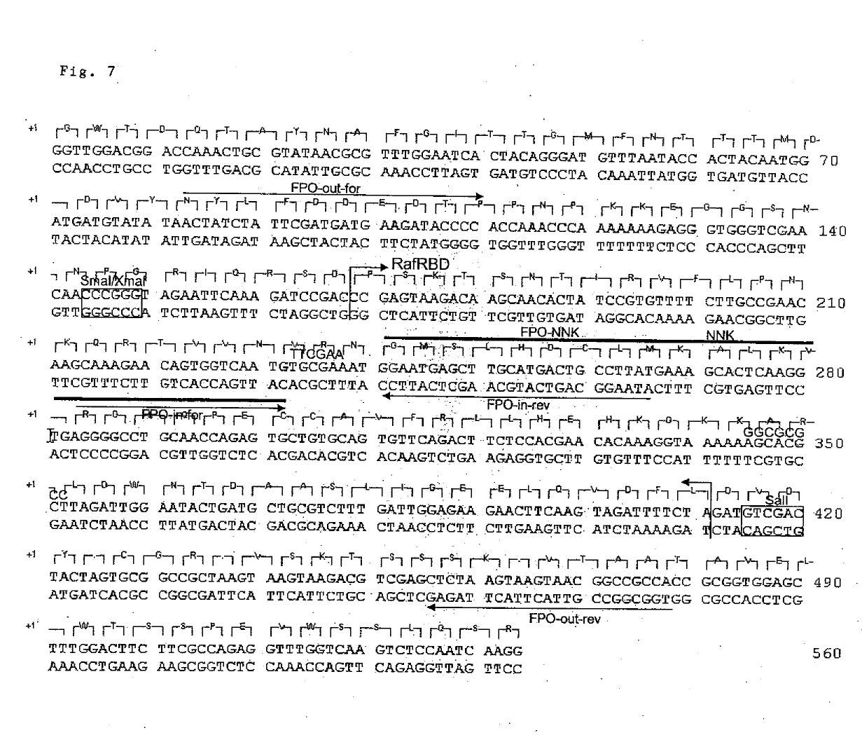

FIG. 7 shows the RafRBD gene with the position of the primers used and of the randomized bridging oligonucleotide. The reverse primers are position below the sequence; the forward primers are positioned above the sequence. The numbering relates to the nucleotide sequence and to the sequences of the restriction cleavage sites which are the result of the silent mutations (see above), and the other cleavage sites used are shown above the sequence.

The primer sequences are as follows:

FPO-out-for
ACTATCTATTCGATGATGAAGATACCCC
FPO-out-rev
TGGCGGCCGTTACTTACTTAGAG
FPO-in-rev
ATAAGGCAGTCATGCAAGCTCATTCC
FPO-in-for
AGGGGCCTGCAACCAGAGTG
FPO-NNK
GGAATGAGCTTGCATGACTGCCTTATGAAANNKCTCAAGGTGAGGGGC
CTGCAACCAGAGTG
FPO-NNK 3
AGAACAGTGGTCAATGTTCGAAATGGAATGAGCTTGCATGACTGCCTT
ATGAAANNKCTCAAGGTGAGGGGCCTGCAACCAGAGTGCTGTGCAGTG
TTCAGACTTCTCCACGAACA

To carry out the PCR, in each case 40 pmol were employed of the primers FPO-out-for and FPO-in-rev (FPOI), and FPO-in-for and FPO-out-rev (FPOII), in a 100 μl PCR mixture for generating the two fragments FPOI and FPOII (see FIG. 4). The templates used were 30 ng of the vector with the silent mutations in the RafRBD gene. The concentration of the dNTPs was 200 μM, and the reaction was carried out in 1×Pfu buffer using 2.4 units of the enzyme. The PCR program was as follows:

94° C. 2 min, 30×(94° C. 30 sec, 60° C. 40 sec, 72° C. 40 sec), 72° C. 5 min

To carry out the in vivo assembly, a total of 500 ng of DNA were introduced into the yeast per transformation reaction, and the vector (cleaved with BstBI/AscI or SmaI/SalI) and fragments/oligo were employed in a molar ratio of 1:4. The fragments FPOI, FPOII and the randomized bridging oligo were transformed.

In all reactions, either pPC97-ras (in the case of the two-hybrid) or pPC97-ras with the competitor TEF promtoer/RafRBD (see FIG. 11) were cotransformed as the second plasmid.

(iii) Robot-Aided Process

The robot-aided process, which involves scanning of the plates, evaluation, hitpicking, DNA isolation from the yeasts and transformation of the plasmids into the bacteria, is shown in detail under item 5.

(iv) Sequencing and Evaluation of the Hits in the Two-Hybrid System

TABLE 4
Hitpicking results

| | |
|---|---|
| FPO I, FPO II, | FPO I, FPO II, |
| FPO-NNK 3 | FPO-NNK 3 |
| vector XmaI/SalI | vector BstBI/AscI |

| Hit | Number | % | Number | % |
|---|---|---|---|---|
| K | 3 | 10.0 | | |
| R | 4 | 13.3 | 4 | 23.5 |
| A | 4 | 13.3 | 2 | 11.1 |
| P | 3 | 10 | 2 | 11.8 |
| G | 3 | 10 | 2 | 11.8 |
| S | 3 | 10 | 3 | 17.6 |
| Others | 2xN, C, V | 33.3 | H, Q, C, N | 5.9 each |
| | 3xH, 1xT 10 | | | |

The fluorophore used was cobA. The restriction enzymes with which the vector used had been cleaved are indicated. The enzyme combination BstBI/AscI generates an overlap region of fragment and vector of 153/130 bp; in the case XmaI/SalI, this overlap region is 64/61 by

The improved mutants are detected in both experiments to a significant degree; in addition to the mutants which are also found in the n-hybrid system (see Table 3), however, additional amino acids are also found. This can be attributed to the fact that the two-hybrid is less selective than the n-hybrid system (see above and author's own data, unpublished).

(v) Sequencing and Evaluation of the Hits in the N-Hybrid System

TABLE 5
Hitpicking results (improved mutants)

FPO I, FPO II,
FPO-NNK
vector BstBI/AscI

| Hit | Number | % |
|---|---|---|
| K | 7 | 87.5 |
| R | 1 | 12.5 |

The fluorophore used was cobA. The restriction enzymes with which the vector used had been cleaved are indicated. The enzyme combination BstBI/AscI generates an overlap region of fragment and vector of 153/130 bp.

The selectivity of the n-hybrid system is drastically increased over that of the two-hybrid system (see Table 3 and author's own data, unpublished); in the improved mutants, it is essentially the mutant with the highest affinity which is identified. The purpose of the generation of a mutagenized library is the detection of improved mutants. Accordingly, the method according to the invention was also employed in the n-hybrid system.

A gap repair may take place. This phenomenon occurs since the wild-type gene is present as a competitor on the second, cotransformed plasmid. To avoid these gap repairs, the gene was synthesized with as much divergence as possible from the original sequence (for example altered codon usage). When employing this gene, recombination should no longer be possible, which leads to a reduced wild-type sequence background. A further possibility of suppressing this process is the use of synthetic wild-type fragments with variability from the wild-type competitor.

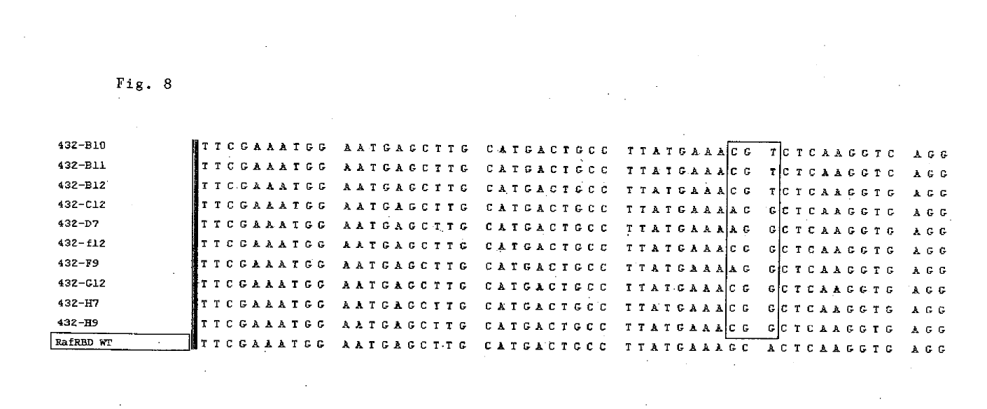

If the codon sequences for arginine are analyzed in a parallel experiment, in which this amino acid had been detected ten times, then a completely equal distribution of the occurring codons is found, in contrast to the bias, described under 2b, of the codons for this amino acid. AGG and CGT occur in each case three times, while CGG occurs four times (see FIG. 8; what is shown is the sequence of part of the RafRBD of 10 clones in comparison with the wild-type sequence (WT=alanine=GCA), the box identifies the randomized position). This result demonstrates that the method according to the invention is suitable for generating a library in which each codon is found with equal frequency.

Double clones (two different codons at the mutagenized position, see above) have not been detected in any of the approaches which were carried out using the method according to the invention. This is an improvement over the approaches with a circular vector or with the fragment generated by in-vivo assembly, where double clones have always been found (see Table 3).

4. Carrying Out the N-Hybrid System

1. Principle of the N-Hybrid Method with Competitor

It is advantageous to express a third component in S. cerevisiae, in addition to the interacting hybrid proteins (fusion proteins) of the known two-hybrid system. If the bait protein is mutagenized, the third component is preferably the free wild-type bait protein. If the prey protein is mutagenized for identifying high-affinity prey proteins, it is preferably the wild-type prey protein which is expressed as competitor.

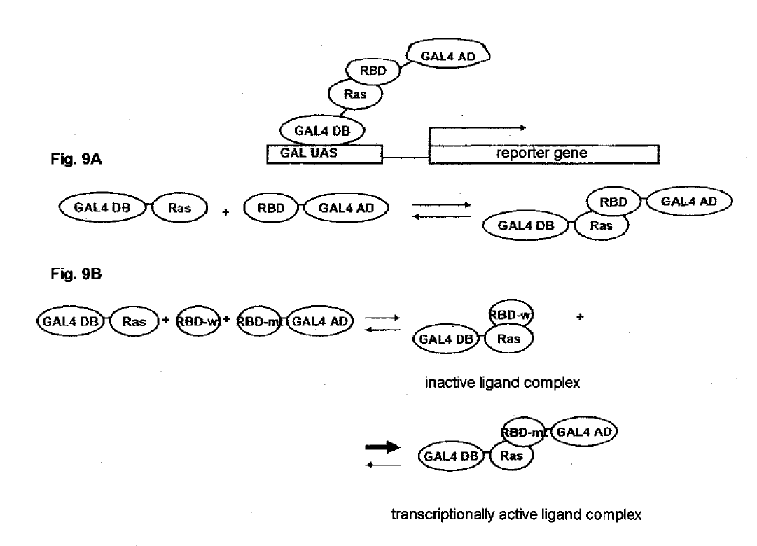

The basic principle of the n-hybrid method in this embodiment is shown in FIG. 9B for the identification of RafRBD prey proteins with affinity, with expression of the wild-type prey protein as competitor. When increasing the affinity of the mutated RasRBD fusion proteins (RBD-mt), the formation of the transcriptionally functional ligand complex is preferred over the nonfunctional (inactive) complex of the competitor RafRBD-wt (RBD-wt) with Ras [abbreviations: RBD-mt: mutated RafRBD fusion protein; RafRBD-wt: wild-type RafRBD; RBD: Ras-binding domain; DB: DNA binding domain; AD: transactivation domain; UAS: upstream activator sequence].

FIG. 9A shows the known two-hybrid system. Only the wild-type variant of the prey protein is employed in the fusion protein in the known system [abbreviations: RBD: Ras-binding domain; DB: DNA binding domain; AD: transactivation domain; UAS: upstream activator sequence].

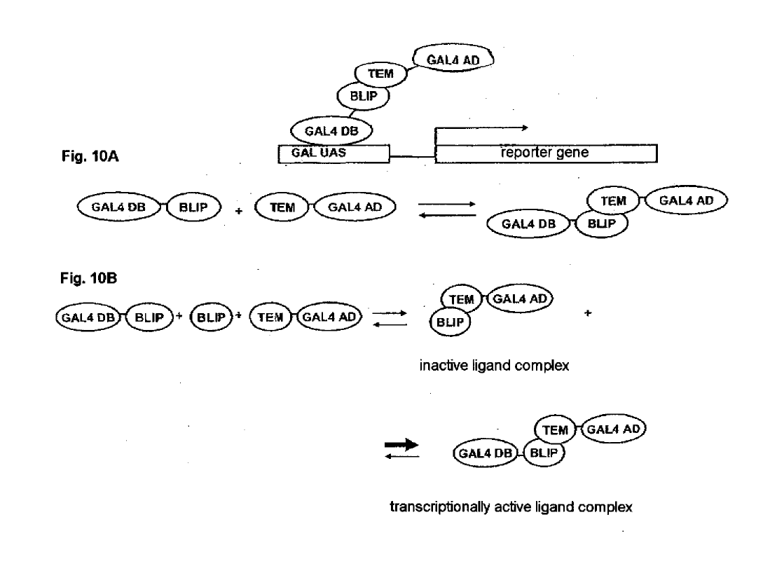

The method according to FIG. 10B serves to study the interaction between the proteins BLIP as bait protein and TEM as prey protein. The prey protein has previously been mutagenized (BLIP-mt) and was employed as the fusion protein. In this system, wild-type BLIP (BLIP-wt) is additionally expressed and acts as competitor. When increasing the affinity of the mutated BLIP fusion protein, the formation of the transcriptionally functional (active) ligand complex is preferred over the inactive ligand complex of BLIP-wt and the fusion protein with TEM [abbreviations: DB: DNA binding domain; AD: transactivation domain; BLIP-wt: wild-type of the bait protein; BLIP-mt: mutated bait protein; UAS: upstream activator sequence]. The Raf-RBD fusion prey proteins had previously been generated via a mutagenesis which is known to the skilled worker. In principle, all customary methods are available for the mutagenesis.

Reporter genes which may be employed for this quantitative screening are, for example, Met1, CysG or CobA (Roessner, C. A. 2002), each of which produces fluorescent derivatives of uroporphyrinogen III. The use of fluorescent proteins as reporter gene is also possible, such as, for example, the use of phycocyanin (Amtz et al., 2004) or RedStar (Knop et al., 2002).

To demonstrate the methodological principle for the combination of the mutagenesis of the prey protein with expression of the wild-type prey protein as competitor, the RafRBD mutants RafRBD-A85K, whose binding affinity has already been characterized in biochemical terms, was employed. According to the author's own unpublished microcalorimetric measurements in PBS buffer, the dissociation constant of the RafRBD-A85K mutant is 72 nM in comparison with dissociation constants of the corresponding RafRBD-wt protein of 253 nM.

When investigating the Ras/Raf interactions in the n-hybrid method, a plasmid derived from the plasmid pPC97 may be used for providing the bait fusion protein Ras-Gal4. The competitor is also encoded on this plasmid. An overview over a possible plasmid is shown in FIG. 11A. FIG. 11B shows a plasmid based on pPC86, which codes for the prey fusion protein.

2. Construction of Yeast Strains for the N-Hybrid Method

### a) Reporter Genes for the Quantitative Screening

The n-hybrid method requires a reporter gene which is under the control of a regulatable promoter and whose activity can be measured directly, qualitatively and quantitatively in the intact yeast colony. Reporter genes which bring about a fluorescence as the readout meet these requirements.

(i) Choice of Promoter for the Reporter Gene

In the yeast Y190, the expression of the reporter gene β-galactosidase is regulated by the strength of the interaction of GAL4 binding and activation domain, whose genes are introduced into the yeast cell by two different vectors (pPC86 and pPC97, Chevray and Nathans, 1992).

The genotype of the yeast strain Y190 is known as: “Mat a, leu2-3, 112, ura3-52, trp1-901, his3-Δ200, ade2-101, gal4Δgal80Δ, URA3::GAL-lacZ, LYS2::GAL-HIS3, cyh^{f}”. Here, URA3::GAL-lacZ means that the promoter in question (GAL; divergent GAL1/GAL10 promoter) has been integrated into the URA3 gene.

The aim of cloning a reporter gene behind this promoter and integrating it into the genome of the yeast requires detailed knowledge of the situation at this position in the genome (cf., in this context, Yocum et al. (1984) for the integration the YIp plasmid pRY171 into the genome of Y152 (derived from YJ0-Z, Leuther and Johnston, 1992), which is the precursor strain of Y153, which, in turn, has given rise to Y190).

Then, the development of the plasmid pRY171, which carries the GAL promoter with the lacZ gene, both downstream of the URA3 gene, was elucidated in order to obtain sequence data: Yocum et al. (1984) have generated this plasmid from the plasmid pLRIΔ3 by removal of the 2 μm replication origin sequences. pLRIΔ3 corresponds to the plasmid pRY131 with the exception of an XhoI linker in the middle of the divergent promoter. pRY131 is generated from West et al. (1984) from pLG 669 (Guarente and Ptashne, 1981) and pRY116. pLG 669, in turn, has originated from YEp24, a plasmid with pBR322 backbone (Botstein et al., 1979).

These data were used to generate a sequence according to which primers (365-for, 394-for, 1563-rev and 1674-rev) were synthetized. These were used to amplify this DNA segment in a PCR with genomic DNA from Y190 and to sequence it. By doing so, the actual sequence of the divergent GAL10/GAL1 promoter, behind which the reporter gene was to be cloned.

Using these primers, the corresponding sections of the promoter were amplified by PCR and then cloned as a fusion product with a fluorophore. In addition, the promoter 365-1451, which no longer contains an lacI/5′lacZ portion, was selected. The reasoning behind this was that additional gene portions might hinder the expression of the selected fluorophore. A further promoter, in which, again, the GAL1 portion was reduced to nil (365-1366) was likewise tested. The fluorophore was RedStar (Knop et al. 2002; see section (iii)).

The best promoter turned out to be the construct 365-1451 (lacI/5′-lacZ no longer present); this construct was employed for all the following integrations of reporter genes into the genome of S. cerevisiae.

Primers Used:

365-for
ACGGGTACCGCAAAGGGAAGGGATGCTAAGG (KpnI)
394-for
ATCGGTACCTGAACGTTACAGAAAAGCAGG (KpnI)
1563-rev
ACTACTAGTGCCTCTTCGCTATTACGCCAGC (SpeI)
1674-rev
AGAACTAGTGGAAGATCGCACTCCAGC (SpeI)
1451-rev
ACAACTAGTAACTTTTCGGCCAATGGTCTTG (SpeI)
1366-rev
ACTACTAGTCCTATAGTTTTTTCTCCTTGACGTTAAA (SpeI)

(ii) FOA Treatment of S. cerevisiae Y190

The n-hybrid method requires reporter genes which are integrated into the genome of the yeast. To this end, a selection marker must be available so that only transformants which have indeed integrated the desired gene into the genome at the correct locus are capable of growing.

The yeast strain Y190 therefore requires an additional marker in addition to the auxotrophism markers leucine and tryptophan, which are confirmed by the two-hybrid system. The marker of choice is uracil (URA3 gene) since this gene offers the possibility of making the strain auxotrophic for this substance.

In the preparation of URA3-negative clones, one exploits yeast's natural mutation frequency of approximately 10⁻⁴. A medium comprising FOA (5-fluoroorotic acid) (Treco DA, 1989) is used in order to be able to select for the mutation events in the yeasts. Yeast cells which no longer produce uracil, that is to say which have the desired phenotype, survive while cells without the mutation in the URA3 gene die (Boeke et al., 1984)).

(iii) RedStar

The marker employed was RedStar (RFP) as a fluorophore which has been optimized for use Saccharomyces cerevisiae (Knop et al., 2002).

The amplification of RedStar was carried out with the following primers:

RedStar-for
ACTACTAGTTATGAGTAGATCTTCTAAGAACGTC (SpeI)
RedStar-rev
TATTCCGCGGTTACAAGAACAAGTGGTGTCTAC (SacII)

The respective promoter and the RedStar gene were cloned into pRS306 as a three-fragment ligation (25 fmol vector, 125 fmol inserts). This vector is an integration vector. The integration of RedStar (or of all other reporter genes under the control of the GAL promoter) into the genome of the uracil-auxotrophic yeast Y190D (see (ii)) can be selected by means of the uracil marker of pRS306.

(iv) cobA

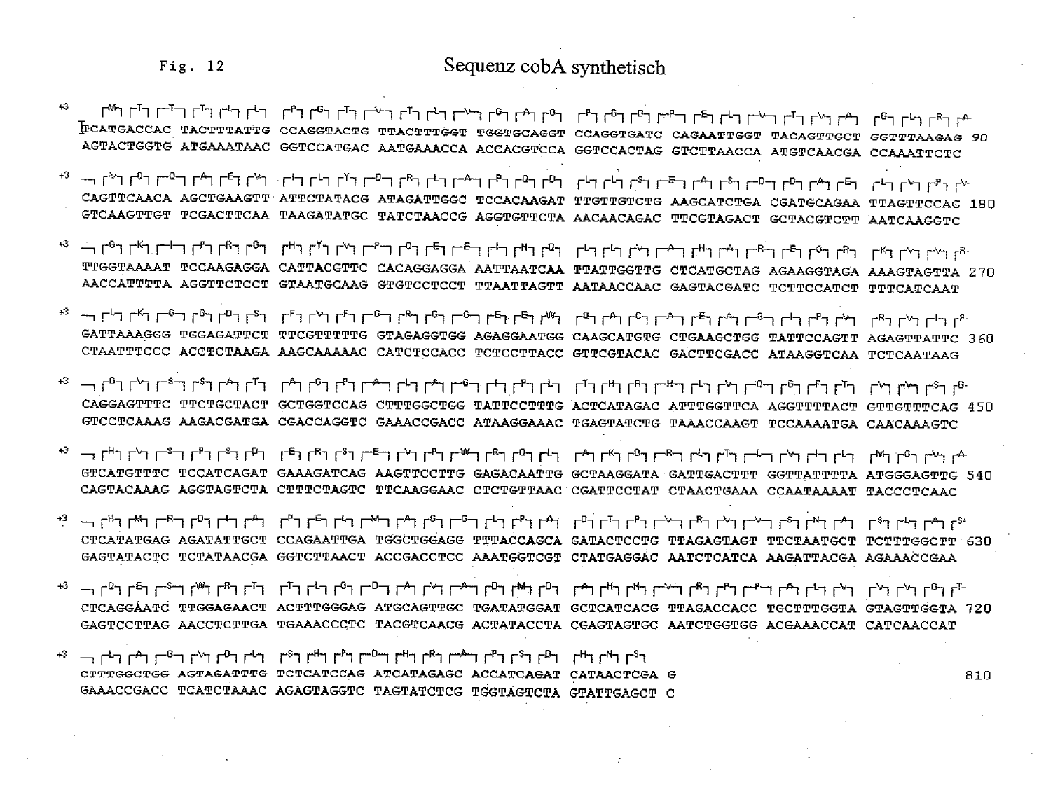

cobA codes for uroporphyrinogen Ill methyltransferase from Propionibacterium freudenreichii. The overexpression of this gene leads to a fluorescence in the range of 605 nm which is due to the accumulation of the fluorescent product trimethylpyrrocorphin (Wildt and Deuschele, 1999). An analysis of the codon usage revealed a high percentage of critical codons during the expression of the bacterial gene in yeast. As a consequence, the sequence was optimized for the frequency of the codon usage of S. cerevisiae and synthesized (sequence see FIG. 12). Again, the gene, once a His tag and a termination sequence (see above) had been added, was cloned together with the promoter of choice (see above) into pRS306, via SacI/NotI, and integrated into the genome of the yeast.

(v) Met1

Met1 is the corresponding protein from Saccharomyces. The 1.8 kb gene (sequence see FIG. 10) was amplified from the yeast genome by means of PCR.

Primers employed:

Met1-for:
AATTATCCATGGTACGAGACTTAGTGACATTG (NcoI)
Met1-rev:
AATTAACTCGAGTTGTATAACTTAAATAGACTATCTACATCAACC (XhoI)

The fragment was cloned via NcoI/XhoI (NcoI contains the start codon) into a vector which permits the addition of His tag and a termination sequence for yeast genes (Arntz et al., 2004). After the reporter gene (NcoI/NotI) together with the promoter of choice (SacI/NcoI) had been cloned into the vector pRS306 via SacI/NotI, the reporter gene was integrated into the genome of the yeast.

### b) Carrying Out the Quantitative Screening in the N-Hybrid Method

Carrying out the quantitative screening involves the preparation of the medium, the transformation of the yeasts and the scanning of the plates. Here, all parameters must be standardized and optimized so that the fluorescence results can be reproduced.

(i) Preparation of the Medium

The following media are required for culturing and scanning the yeasts for fluorescence with the LSA scanner:
YPAD medium (complete medium for yeasts)
5.0 g Yeast extract (Difco)
10.0 g Peptone (Difco)
50 mg Adenine hemisulphate
ddH₂O to 460 ml
Before autoclaving, bring pH to 5.8; after autoclaving, the medium has a pH of 5.6; for agar plates: addition of 10 g Yeast agar (Difco) after the pH has been adjusted Autoclave for 15 minutes at 121° C.;
after autoclaving, 40 ml of 25% strength glucose (autoclave separately from the medium) is added.

Synthetic Full Medium (without Leu, Trp, His)
3.35 g Yeast Nitrogen Base (w/o amino acids)
1 g Synthetic Complete prop Out Mix (amino acid mix without Leu, Trp, H is)
ddH₂O to 460 ml;
before autoclaving, bring pH to 5.8; after autoclaving, the medium has a pH of 5.6; for agar plates: addition of 10 g Yeast agar (Difco) after the pH has been adjusted Autoclave for 15 minutes at 121° C.;
after autoclaving, 40 ml of 25% strength glucose (autoclaved separately from the medium) and 10 ml of 2.5 M 3-amino-1,2,4-triazole (filter-sterilized) are added.
Final glucose concentration in the medium: 2%
25% glucose
100 g glucose (Sigma)
400 ml ddh₂O
Autoclave for 15 minutes at 121° C.
2.5 M 3-amino-1,2,4-triazole
1.051 g 3-amino-1,2,4-triazole
5 ml ddH₂O
Filter-sterilize using a sterile filter (diameter 0.45 μm); the final 3-amino-1,2,4-triazole concentration in the medium depends on the experiment and is between 0 and 50 mM.

Ominitray plates (from Nunc), on which the yeasts for scanning in the LSA scanner are cultured, are poured in a standardized manner with a volume of 78 ml of medium. This results in always the same scanning height for the scanner, which is adjusted to 9.9 mm.

(ii) Performing the Yeast Transformation

The following protocol, which is optimized for the maximum transformation efficiency, is employed.

20-30 ml of liquid YPAD (complete medium) are inoculated with the yeasts to be transformed (Y190D with integrated reporter gene) and incubated overnight at 30° C. and 200 rpm. On the next day, yeasts from the preculture are pipetted to 50 ml of YPAD (brought to room temperature) until an OD₆₀₀ of approximately 0.05 has been reached. The culture is incubated at 30° C. and 150-200 rpm until a cell density of 2×10⁶−4×10⁶ cells/ml has been reached. This corresponds to an OD₆₀₀ of 0.2-0.4 (takes approximately 3-5 h). The culture is harvested in a sterile 50-ml centrifuge tube at 3000×g (3500 rpm in the Hettich centrifuge) and 5 minutes. The medium (supernatant) is removed and the cells are resuspended in 25 ml of sterile ddH₂O. After resuspending, the cells are again centrifuged for 5 minutes at 3000×g (3500 rpm Hettich centrifuge). The supernatant is removed and the cells are resuspended in 1.0 ml of 100 mM LiAc. The suspension is transferred into a 1.5 ml Eppendorf cup. The cells are now incubated for 15 minutes at 30° C. The cells are subsequently pelleted by centrifugation at full speed for 15 seconds and the supernatant is drawn off. This amount of cells is sufficient for one transformation reaction. If two transformation reactions are to be performed, 100 ml (2×50 ml) of competent cells must be prepared and pretreated with lithium acetate. The following transformation mix is pipetted to the cells, in the order stated:
X μl plasmid DNA (0.1-10 μg)
34-X μl sterile ddH₂O
(Cells in water+plasmid solution resuspend by pipetting up and down, only then add PEG, using a pipette)
240 μl PEG (50% w/v)
(Mix Cells and Peg by Briefly Vortexing)
36 μl 1.0 M LiAc
50 μl ss DNA (2.0 mg/ml)
total volume 360 μl

The cells are vortexed vigorously until a homogeneous suspension has formed (approx. 1 min). The transformation reaction is incubated for 30 minutes in a shaker (800 rpm) at 30° C. and subsequently placed into a waterbath at 42° C. (heat shock). After the incubations, the transformation reaction is centrifuged for 15 seconds at 6-8000 rpm and the transformation mix is removed from the Eppendorf cup using an Eppendorf pipette. The pellet (cells) are treated with 1.0 ml of sterile ddH₂O and the pellet is resuspended by slowly pipetting up and down. Rapid pipetting up and down reduces the transformation efficiency. The dissolved transformed cells are diluted once 1:100 and once 1:10 000, and between 2 and 200 μl of the dilute cells are streaked onto SC medium without leucine, tryptophan and histidine, but with a suitable concentration of 3-aminotriazole. With a plated volume of 20-200 μl and a dilution of 1:10 000, the number of colonies to be expected is 0-50 colonies per plate, and with a plated volume of 10-200 μl with a dilution of 1:100 to 200→5000 colonies per plate. In this case, the transformation efficiency is 500 000-2 000 000 cells per μg of plasmid DNA (the transformation efficiency decreases with increasing 3-AT concentration). The plates are incubated for 2-6 days at 30° C.

After 2-6 days (depending on the interaction pair and 3-AT concentration), the yeast cells can be scanned and evaluated in the LS-400 scanner (Tecan).

(iii) Scanning with the Tecan LS-400

The hardware (the Tecan LS-400 scanner) together with the matching software can be obtained from Tecan.

The height of the agar (scanning height) of the Omnitray plate is at least 8.0 mm.

Existing methods are applied to the measurements. Clones with RedStar, cobA or Met1 in the genome and two-hybrid or n-hybrid plasmids are scanned with the laser 543 nm and the filter 590 nm (bandpass 20 nm).

The images are taken in the non-confocal mode.
The scan resolution when scanning a normally grown culture (diameter approx. 1-2 mm) is set at 20 μm. If a culture consists of smaller colonies, the scan resolution is reduced to 4 to 8 μm.

(iv) Evaluation of the Scanned Colonies with Optimate

The measurements are evaluated with the software Optimate, which has been developed for this application in cooperation and is commercially available from Tecan.

The following settings are optimized for a culture which consists of colonies with a diameter of 1-2 mm:

| | |
|---|---|
| Minimum Object: | 70 |
| Roundness: | 15.2 |
| Threshold Power: | 15 |

All colonies which are present individually and which are large enough are evaluated. The fluorescence intensity is standardized for the area.

### c) Cloning the Competitor

(i) Choice of Different Promoters for the Competitor—Preliminary Test

The concentration of the competitor is essential for the n-hybrid system. The more gene product present, the more the equilibrium shifts towards the side of the inactive complex of competitor and fusion bait protein.

To ensure variable concentrations of the competitor, one should employ various promoters which have been described in the literature (Nacken et al., 1996) as constitutive promoters for expressing the competitor and validate them in the yeast strain.

KEX2 (Fuller at at. 1989, M24201), 488 bp
KEX2-for:
ATCCTTGAGCTCTCAGCAGCTCTGATGTAGATACAC (SacI)
KEX2-rev:
ATCCCCCATGGCTGATAATGGGTTAGTAGTTTATAATTATGTG (NcoI)
TEF (Cottrelle at al., 1985, M10992) 411 bp
TEF-for:
ATCCCCGCGGTAGCTTCAAAATGTTTCTACTCC (SacII)
TEF-rev:
ATCCCCCATGGTTTGTAATTAAAACTTAGATTAGATTG (NocI)
GAPDH (Bitter and Egan, 1984, M13807), 13,680 bp
GAPDH-for:
ATCCCCGCGGCAGTTCGAGTTTATCATTATCAATAC (SacI)
GAPDH-rev:
ATCCCCCATGGTTTGTTTGTTTATGTGTGTTTATTC (NcoI)

These promoters were amplified with the stated primers from the yeast genome (SacII or SacI/NcoI), cloned together with the RedStar gene (BspHI/NotI) into pRS306 (SacII or SacI/NotI) and integrated into the yeast genome.

In the following step, all three promoters were cloned upstream of the competitor (see section which follows).

(ii) Cloning the Prey Protein Competitor on the Fusion Prey Protein Plasmid

To carry out the method according to the invention, the competitor is cloned into one of the two two-hybrid plasmids and in this manner ideally synthesized by the cell itself, as are the bait and prey protein. If the prey protein is to be used as the competitor, it is cloned onto the vector with the fusion bait protein; if the bait protein is to be present as the competitor, it is cloned onto the vector with the fusion prey protein. This avoids possible recombination reactions between identical gene sequences which may take place in the yeast.

The cloning of RafRBD (prey protein competitor) onto pPC97-ras (fusion bait protein plasmid) is to be described by way of use example.

The following structure exists in pPC97-ras:

Promoter (ADH)-GAL4-BD-ras-mcs (AatII/SacI/SacII)-terminator (ADH)

In order to be able to clone the competitor in, a terminator must be introduced downstream of the ras gene; this should be followed by the promoter and then by the competitor. To this end, one uses the terminator which is also attached to other genes to be cloned (see Arntz et al., 2004). In this case, two oligos are annealed (term-Raf-for and—rev; sequence see hereinbelow). To this end, the oligos are annealed in a final concentration of in each case 2 pmol/μl using a PCR machine (94° C. 2 min, 70×−1° C., in each case 1 min at this temperature, 4° C.; instructions from Pierce: Anneal complementary pairs of oligonucleotides, Technical Resource). 2 μl are employed for the subsequent ligation into the vector.

Cloning into the fusion bait protein plasmid is effected via AatII/SacI. The RafRBD gene is amplified (PciI/SacII) and together with the respective promoter of choice (SacI/NcoI) cloned, as a three-fragment ligation, into the vector with terminator (SacI/SacII). The structure shown in FIG. 11a results.

Primers used:

Term-Raf-for
CTATATAACTCTGTAGAAATAAAGAGTATCATCTTTCAAAGAGCT
Term-Rakev:
CTTTGAAAGATGATACTCTTTATTTCTACAGAGTTATATAGACGT
RafRBD-Pci-for:
AATTCCACATGTCCGACCCGAGTAAGACAAGC (PciI)
RafRBD-SaciI-rev:
ATTGCCGCGGTTAGTCGACATCTAGAAAATCTACTTGAAG (SacII)

5. Performing the Robot-Aided Hitpickinq

(i) Hitpicking using the Tecan Freedom 200

Both hardware and software are commercially available from Tecan; the software was developed in cooperation.

The program Gemini performs the script “Colony-Pick”. The % hits to be picked is input. 70% strength ethanol is provided in the container “Steril 1” for sterilizing the pipetting and picking canulas. The picked colonies are deposited in microtiter plates which contain the same selection medium (SC-LWH agar for the selected yeast cultivation in the two-hybrid and the n-hybrid) as the medium on which the yeasts to be picked were cultured.

The Software Facts perform the process ColonyPicking; here, a method of how the scanning is to be performed may be selected. The method is fixed for Colony-Pick (Gemini); the method “Redstar-Scanning” must be carried out for clones with the Redstar or the cobA gene as the reporter gene, with the following settings:
Scan Area: Top 73 mm, Left 2 mm, Bottom 2 mm, Right 114 mm
Autofocus: Z-Scan End 1600 μm, Z-Scan Start 1600 μm
Focus Offset: 0 μm, Focal Plane: Plane 1
Laser: 543 nm, Filter: 590 nm, Scan Resolution: 20 μm, Pinhole: Large

The Omnitray plates must be provided with a bar code.

After the colonies have been picked and cultured for 2 days at 30° C. in the incubator, the plasmids are reisolated from the yeasts and sequenced after having been transformed into bacteria.

(ii) DNA Isolation from Yeasts with the Tecan T-Mags

After the yeasts had been cultured for 2 days in the microtiter plates, the DNA may be isolated from the yeasts. In a deep-well plate, 1000 μl of medium (SC-LWH) are pipetted into each well. The colonies are now inoculated from the Resource plate (plate on which the yeast colonies grow after picking) into the deep-well plate containing the respective selection medium. Approximately 200 μl of medium (SC-LWH)/well is pipetted to the yeasts in the microtiter plate, and they are resuspended by repeatedly pipetting up and down. The resuspended yeasts are subsequently transferred into the deep-well plate into which medium had previously been introduced. The yeasts are now incubated on a microtiter plate shaker for approximately 16 hours at 30° C.

On the next day, the optical density of some wells is determined by adding 100 μl of these cells from one well to 900 μl of medium. The optical density of this 10-fold dilution is then determined. Using these data, the mean of all wells is determined. The deep-well plate with the cells is then centrifuged for 5 minutes in a swinging-bucket rotor (Sorvall centrifuge) at 2500 rpm. Another deep-well plate is filled with the same volume of H₂O to act as counterweight. After centrifugation, the supernatant is removed from the deep-well plate by decanting. 300 μl of Y1 buffer are pipetted into each well. In addition, 1-2 units Lyticase/OD₆₀₀ (of the yeasts in the wells) are pipetted into each well. Buffer and Lyticase are mixed thoroughly with the cells. The deep-well plate is incubated for 1.5 hours at 30° C. (do not shake). The deep-well plate is centrifuged in the swinging-bucket rotor (Sorvall centrifuge) for 5 minutes at 2500 rpm. After the centrifugation, the supernatant is removed from the deep-well plate by decanting. The cells in the deep-well plate are taken up in 250 μl of ddH₂O. The cells must be resuspended thoroughly. The deep-well plate is now ready for the DNA isolation.

The Gemini software of the Tecan robot is used to carry out a DNA isolation method which has been developed by AGOWA (Berlin) in collaboration with Tecan. This isolation takes place in the T-Mags apparatus on the robot platform.

(iii) Transformation of Bacteria

Preparation of Competent Bacteria for Transformation in PCR Plates

5 ml of SOB medium are inoculated with an individual colony of E. coli DH5alpha bacteria. The cells are incubated overnight in a shaker (210-225 rpm) at 37° C. 50-100 μl of this culture are transferred into 100 ml of SOB medium and incubated in the shaker (180 rpm) at 37° C. The bacteria are harvested at OD₆₀₀=0.1 to 0.5 (after approx. 2-3 hours) and placed on ice for 20 minutes. From now on, all further steps are carried out at a temperature of 4° C. The bacterial culture is centrifuged in a 50 ml Falcon vessel (conical bottom) at 4° C. and 1200 g. The pellet is resuspended in 10 ml of ice-cold 50 mM CaCI2 solution by pipetting up and down and subsequently incubated on ice for at least 30 minutes. The cells are now centrifuged for 5 minutes at 4° C. and 1200 g. The cells are resuspended in 1 ml/0.1 OD₆₀₀ ice-cold 50 mM CaCI2 solution with 15% glycerol by pipetting up and down. At an OD₆₀₀=0.1, the cells are therefore taken up in 1 ml, at an OD₆₀₀=0.3 in 3 ml, of CaCI2. 10 μl portions per well are transferred to a PCR plate which has been precooled on ice and frozen at −80° C. and stored.

Transformation of the Bacteria with the DNA from Yeasts

Defrosting of the competent bacteria in the PCR plates was performed in a PCR block made of metal and resting on ice. 10 μl of isolated DNA are pipetted into each well. Bacteria and DNA are mixed carefully (do not pipette up and down!). The cells are now incubated on ice for at least 30 min. Then, a heat shock of 30 s at 42° C. is carried out, using a heating block. After the heat shock, the cells are returned to ice for 2 min. 100 μA of SOC medium are introduced into a deep-well plate. 100 μl of SOC per well are also pipetted to the bacteria. The bacteria are transferred from the PCR plate into the deep-well plate and incubated into the microtiter plate shaker for 1 hour at 37° C. at 210 to 225 rpm. After incubation for 1 hour, 1 ml of LB medium with the suitable antibiotic is pipetted to the cells and the plates are incubated in the microtiter plate shaker for at least 20 hours at 37° C. at 210 to 225 rpm. After this incubation, 5-10 μl of the cells are transferred to a 96-well plate with LB agar+antibiotic, and this plate is incubated in the incubator for 16 hours at 37° C. This plate may be sent to AGOWA to have the individual colonies sequenced.

6. Random Mutagenesis

Mutagenesis on RafRBD A85

(i) Construction of the Necessary Vectors

After the transformation of the mutagenized library, there are two plasmids in the yeast, both of which carry an ampicillin resistance gene. Firstly, pPC97 with the ras gene (in the n-hybrid system, this plasmid additionally contains the competitor; see FIG. 11) and, secondly, pPC86, by which the mutated RafRBD gene is encoded. After the transformation, into competent bacteria, of the DNA isolated from these yeasts, only the plasmid on which the mutated RafRBD gene is present should be present in the bacteria. To this end, one of the vectors must be equipped with a different antibiotic resistance. In the present case, pPC86 was provided with a kanamycin resistance. For this purpose, a PmeI site was generated in each case upstream and downstream of the TEM resistance gene by means of QuikChange mutation, following the manufacturer's instructions, and the gene was then excised and replaced by the kanamycin resistance gene. The bacteria which had been transformed with DNA from yeast will now grow in kanamycin-containing medium and can thus be separated from the bacteria which contain the fusion bait protein plasmid pPC97.

Primers used:

Multi-QC-Pme-upstream-TEM
TGAATACTCATACTCTTCCTGTTTAAACATTATTGAAGCATTTATCA
GGG
Multi-QC-Pme-downstream-TEM:
TTAAATCAATCTAAAGTATATATG7TTAAACTrGGTCTGACAGTTAC
CAATG
(PmeI)
Pme-Kan-for:
AAAAAACCGTTTAAACAGGAAGAGTATGATTGAACAAGATGGATTGC
(PmeI)
Pme-Kan-rev:
AAAAAACCGTTTAAACTTGGTCTGACAGTCAGAAGAACTCGTCAAGA
AGG
(PmeI)

The random mutagenesis is carried out by the method of Zheng et al. (2004) (see section b). To this end, the following two primers were designed, which partially overlap and which randomize the amino acid A85 of RafRBD:

Z-RBD-A85-fort:
CTGCCTTATGAAANNKCTCAAGGTGAGGGGOCTGCAACCAG
Z-RBD-A85-rev
CCCTCACCTTGAGMNNTTTCATAAGGCAGTCATGCAAGCTC

These primers are employed for the PCR using the Expand kit (Roche). To this end, 50 ng of template DNA (pPC86-RafRBD with kanamycin resistance gene) were used, and the PCR was carried out with 0.8 pmol/μl of each primer following the manufacturer's instructions. The PCR reaction is then purified with the PCR Purification kit (Qiagen), and 5 of 50 μl are applied to an agarose gel. The remainder of the mixture is restricted for 3 hours at 37° C. with 10 U DpnI (NEB, in buffer 4) in order to remove the template DNA, which is methylated since it has been isolated from Emil. Zheng et al. carried out the digestion only for 1 hour, but this resulted in a high background of wild-type clones in the library.

Then, 2.5 μl of the DpnI cut are transformed into 75 μl of competent XL10 Gold cells (Stratagene) following the manufacturer's instructions; addition of 750 μl of NZY after the heat shock is followed by a one-hour regeneration phase of the bacteria. 20 μl of the transformation mixture are plated (approximately 1/20 of the total mixture) for determining the transformation efficiency, while the remainder is incubated overnight at 37° C. and 225 rpm on the shaker in LB medium with kanamycin. The DNA is isolated the next morning. The number of presumably independent colonies is determined by counting the plated colonies and extrapolation to the total number (factor 20).

The library is characterized by sequencing single colonies and the library DNA. This DNA is then transformed into the yeast which contains a reporter gene in the genome. Here, firstly pPC97-ras (for the two-hybrid) and secondly pP97-ras with competitor TEF promoter/RafRBD (for the n-hybrid system) is employed as the second plasmid.

7. Use of Forward- and Reverse-Randomized Oligonucleotides

The use of double-stranded oligonucleotides results in a high transformation efficiency. Surprisingly, the simultaneous use of two single-stranded oligonucleotides in the opposite orientation resulted in a comparable transformation rate, the hitpicking of the improved mutants being at least equally good.

TABLE 6

NNK2 for and rev, 300 ng
26 colonies

| Hit | Number | % |
|---|---|---|
| K | 6 | 23.1 |
| R | 16 | 61.5 |
| A | | |
| P | 3 | 11.5 |
| G | 1 | 3.8 |
| S | | |
| Remainder | | |

### REFERENCES

Arntz, C., Meinders, D., Block, C., Mittmann, K (2004) Phycocyanin-expressing eukaryotic cell EPA 04 001 504.2
Bitter G A and Egan KM (1984) Expression of heterologous genes in Saccharomyces cerevisiae from vectors utilizing the glyceraldehydes-3-phosphate dehydrogenase gene promoter. Gene 32:263-274
Block C., Janknecht R., Herrmann C., Nassar N., Wittinghofer A. (1996) Quantitative structure-activity analysis correlating Ras/Raf interaction in vitro to Raf activation in viva. Nature Structural Biology 3:244-251
Boeke J D, LaCroute F, Fink GR. (1984) A positive selection for mutants lacking orotidine-5′-phosphate decarboxylase activity in yeast: 5-fluoro-orotic acid resistance. Mol. Gen. Genet. 197(2):345-6
Bosley A D, Ostermeier M (2005) Mathematical expressions useful in the construction, description and evaluation of protein libraries. Bimolecular Engineering 22:57-61
Botstein D, Falco S at al. (1979) Sterile host yeasts (SHY): A eukaryotic system of biological containment for recombinant DNA experiments. Gene 8:17-24
Cottrelle P, Thiele D, Price V, Memet S, Micouin J-Y, Merck C, Buhler J-M, Sentenac A, Fromageot P (1985) Cloning, Nucleotide Sequence, and Expression of One of Two Genes Coding for Yeast Elonation Factor 1a. JBC 260:3090-3096
Fridman, M., Marula, H., Gonez, J., Walker, F., Treutlein, H., Zeng, J. and Burgess, A. (2000) Point Mutants of c-raf-1 RED with elevated Binding to v-Ha-Ras. J. Biol. Chem. 275, 30363-30371
Fusco C, Guidotti E, Zervos AS (1999) In vivo Construction of cDNA Libraries for Use in the Yeast Two-Hybrid System. Yeast 15:715-720
Fuller R S, Brake A, Thorner J (1989) Yeast prohormone processing enzyme (KEX2 gene product) is a Ca 2+-dependent serine protease. PNAS 86:1434-1438
Guarante L. Ptashne M (1981) Fusion of Escherichia coli lacZ to the cytochrome c gene of Saccharomyces cerevisiae. PNAS 78:2199-2203
Hogrefe H H, Cline J, Youngblood G L, Alien R M (2002) Creating Randomized Amino Acid Libraries with the QuikChange Multi Site-Directed Mutagenesis Kit. Bio Techniques 33(5):1158-1165
Hue S, Luo Y, Qiu M, Chan E, Zhou H, Zhu L (1998) Construction of a modular yeast two-hybrid cDNA library from human EST clones for the human genome protein linkage map. Gene 215:143-152
Jaitner, B. K., Becker, J., Linnemann, T., Herrmann, C., Wittinghofer, A. and Block C. (1997) Discrimination of Amino Acids mediating Ras binding from noninteracting Residues affecting Raf Activation by Double Mutant Analysis. J. Biol. Chem. 272, 29927-29933
Knop, M., Barr, F., Riedel, C G., Reichel, C. (2002) Improved Version of the Red Fluorescent Protein (drFP583/DsRed/RFP). Bio Techniques 33:592-602
Leuther K K, Jonston S A (1992) Nondissociation of GAL 4 and GAL 80 in Vivo After Galactose Induction. Science 256:1353-1335
Muhlrad D, Hunter R, Parker R (1992) A Rapid Method for Localized Mutgenesis of Yeast Genes. Yeast 8:79-82
Nacken V. Achsletter T. Degryse E (1996) Probing the limits of expression levels by varying promoter strength and plasmid copy numer in Saccharomydes cerevisiae. Gene 175:2563-260
Palfrey D, Picardo M, Hine A V (2000) A new randomization assay reveals unexpected elements of sequence bias in model “randomized” gene libraries: implications for biopanning. Gene 251:91-99
Roessner C. A. (2002) Use of cobA and cysGA as red fluorescent indicators. Methods Mol. Biol. 183, 19-30
Schaerer-Brodbeck C, Barberis A (2004) Coupling homologour recombination with growth selection in yeast a tool for construction of random DNA sequence libraries. Bio Techniques 37:202-206
Sneeden J L, Loeb L A (2003) Random Oligonucleotide Mutagenesis. In: Methods in Molecular Biology, Vol. 231:65-73. Edited by F H Arnold and G. Georgiou. Humana Press
Stemmer WPC (1994) DNA shuffling by random fragmentation and reassembly: in vitro recombination for molecular evolution. PNAS 91:10747-10751
Treco DA Basic Techniques of Yeast Genetics (1989) In: Current Protocols in Molecular Biology, Wiley, Supplement 5. Unit 13.1, p 13.1.5
West R W, Yocum R R, Ptashne M. (1984) Saccharomyces cerevisiae GAL1-GAL10 Divergent Promoter Region: Location and Function of the Upstream Activating Sequence UAS_{G} Mol Cell Biol November 1984:2467-2478
Wildt S and Deuschle U (1999) cobA, a red fluorescent transcriptional reporter for Escherichia Coli, yeast, and mammalian cells. Nat Biotech 17:1175-1178
Yocum R R, Hanley S, West R, Ptashne M (1984) Use of lacZ Fusions to Delimit Regulatory Elements of the Inducible Divergent GAL1-GAL10 Promoter in Saccharomyces cerevisiae. Mol Cell Biol October 1984:1985-1998
Zheng L, Baumann U, Reymond J-L (2004) An efficient one-step site-directed and site-saturation mutagenensis protocol. Nucleic Acids Research 32 (14): e115

1-9. (canceled)

10. A method of generating genetic diversity in vivo, comprising the following steps:
generating fragments of at least one wild-type gene sequence, wherein the fragments have in each case at least one region which is homologous with a vector suitable for the in-vivo recombination;
generating randomized bridging nucleotides of at least one defined oligonucleotide sequence, wherein parts of the bridging oligonucleotide are homologous with at least one fragment of the wild-type gene sequence, and the bridging oligonucleotides are randomized via non-PCR-based methods, and
introducing the linearized vector, at least one wild-type fragment and the randomized bridging oligonucleotides into an in-vivo system for homologous recombination.

11. The method as claimed in claim 1, further comprising the step of cloning a wild-type-heterologous DNA segment, which is a stuffer fragment, into the wild-type sequence.

12. The method as claimed in claim 1, wherein a host organism is transformed with at least four wild-type fragments, wherein only two have a sequence region with homology to the vector.

13. The method as claimed in 12. wherein the host organism is transformed with more than one randomized bridging oligonucleotide.

14. The method as claimed in claim 13, wherein the transformation is carried out in yeast.

15. The method as claimed in claim 14, wherein a selection of the transformed yeast clones is performed via a two-hybrid or an n-hybrid system.


SEQUENCE LISTING (35 sequences)

SEQ ID NO: 1 - DNA, 37 bp, artificial
  Primer QC-RafRBD-BstBI
aagaacagtggtcaatgttcgaaatggaatgagcttg

SEQ ID NO: 2 - DNA, 48 bp, artificial
  Primer QC-RafRBD-AscI
acgaacacaaaggtaaaaaggcgcgcctagattggaatactgatgctg

SEQ ID NO: 3 - DNA, 28 bp, artificial
  Primer FPO-out-for
actatctattcgatgatgaagatacccc

SEQ ID NO: 4 - DNA, 23 bp, artificial
  Primer FPO-out-rev
tggcggccgttacttacttagag

SEQ ID NO: 5 - DNA, 26 bp, artificial
  Primer FPO-in-rev
ataaggcagtcatgcaagctcattcc

SEQ ID NO: 6 - DNA, 20 bp, artificial
  Primer FPO-in-for
aggggcctgcaaccagagtg

SEQ ID NO: 7 - DNA, 62 bp, artificial
  Primer FPO-NNK
ggaatgagcttgcatgactgccttatgaaannkctcaaggtgaggggcctgcaaccagagtg

SEQ ID NO: 8 - DNA, 116 bp, artificial
  Primer FPO-NNK 3
agaacagtggtcaatgttcgaaatggaatgagcttgcatgactgccttatgaaannkctcaaggtgaggggcctgcaaccagagtgctgtgcagtgttcagacttctccacgaaca

SEQ ID NO: 9 - DNA, 31 bp, artificial
  Primer 365-for
acgggtaccgcaaagggaagggatgctaagg

SEQ ID NO: 10 - DNA, 30 bp, artificial
  Primer 394-for
atcggtacctgaacgttacagaaaagcagg

SEQ ID NO: 11 - DNA, 31 bp, artificial
  Primer 1563-rev
actactagtgcctcttcgctattacgccagc

SEQ ID NO: 12 - DNA, 27 bp, artificial
  Primer 1674-rev
agaactagtggaagatcgcactccagc

SEQ ID NO: 13 - DNA, 31 bp, artificial
  Primer 1451-rev
acaactagtaacttttcggccaatggtcttg

SEQ ID NO: 14 - DNA, 37 bp, artificial
  Primer 1366-rev
actactagtcctatagttttttctccttgacgttaaa

SEQ ID NO: 15 - DNA, 34 bp, artificial
  Primer RedStar-for
actactagttatgagtagatcttctaagaacgtc

SEQ ID NO: 16 - DNA, 33 bp, artificial
  Primer RedStar-rev
tattccgcggttacaagaacaagtggtgtctac

SEQ ID NO: 17 - DNA, 781 bp, artificial
  Sequence cobA (orininally stemming from Propionibacterium freudenreichii) optimized for the frequentness of codon usage in S. cerevisiae
tcatgaccactactttattgccaggtactgttactttggttggtgcaggtccaggtgatccagaattggttacagttgctggtttaagagcagttcaacaagctgaagttattctatacgatagattggctccacaagatttgttgtctgaagcatctgacgatgcagaattagttccagttggtaaaattccaagaggacattacgttccacaggaggaaattaatcaattattggttgctcatgctagagaaggtagaaaagtagttagattaaagggtggagattctttcgtttttggtagaggtggagaggaatggcaagcatgtgctgaagctggtattccagttagagttattccaggagtttcttctgctactgctggtccagctttggctggtattcctttgactcatagacatttggttcaaggttttactgttgtttcaggtcatgtttctccatcagatgaaagatcagaagttccttggagacaattggctaaggatagattgactttggttattttaatgggagttgctcatatgagagatattgctccagaattgatggctggaggtttaccagcagatactcctgttagagtagtttctaatgcttctttggcttctcaggaatcttggagaactactttgggagatgcagttgctgatatggatgctcatcacgttagaccacctgctttggtagtagttggtactttggctggagtagatttgtctcatccagatcatagagcaccatcagatcataactcgag

SEQ ID NO: 18 - DNA, 32 bp, artificial
  Primer Met1-for
aattatccatggtacgagacttagtgacattg

SEQ ID NO: 19 - DNA, 45 bp, artificial
  Primer Met1-rev
aattaactcgagttgtataacttaaatagactatctacatcaacc

SEQ ID NO: 20 - DNA, 36 bp, artificial
  Primer KEX2-for
atccttgagctctcagcagctctgatgtagatacac

SEQ ID NO: 21 - DNA, 43 bp, artificial
  Primer KEX2-rev
atcccccatggctgataatgggttagtagtttataattatgtg

SEQ ID NO: 22 - DNA, 33 bp, artificial
  Primer TEF-for
atccccgcggtagcttcaaaatgtttctactcc

SEQ ID NO: 23 - DNA, 38 bp, artificial
  Primer TEF-rev
atcccccatggtttgtaattaaaacttagattagattg

SEQ ID NO: 24 - DNA, 36 bp, artificial
  Primer GAPDH-for
atccccgcggcagttcgagtttatcattatcaatac

SEQ ID NO: 25 - DNA, 36 bp, artificial
  Primer GAPDH-rev
atcccccatggtttgtttgtttatgtgtgtttattc

SEQ ID NO: 26 - DNA, 45 bp, artificial
  Primer Term-Raf-for
ctatataactctgtagaaataaagagtatcatctttcaaagagct

SEQ ID NO: 27 - DNA, 45 bp, artificial
  Primer Term-Raf-rev
ctttgaaagatgatactctttatttctacagagttatatagacgt

SEQ ID NO: 28 - DNA, 32 bp, artificial
  Primer RafRBD-Pci-for
aattccacatgtccgacccgagtaagacaagc

SEQ ID NO: 29 - DNA, 40 bp, artificial
  Primer RafRBD-SacII-rev
attgccgcggttagtcgacatctagaaaatctacttgaag

SEQ ID NO: 30 - DNA, 50 bp, artificial
  Primer Multi-QC-Pme-vor-TEM
tgaatactcatactcttcctgtttaaacattattgaagcatttatcaggg

SEQ ID NO: 31 - DNA, 52 bp, artificial
  Primer Multi-QC-Pme-nach-TEM
ttaaatcaatctaaagtatatatgtttaaacttggtctgacagttaccaatg

SEQ ID NO: 32 - DNA, 47 bp, artificial
  Primer Pme-Kan-for
aaaaaaccgtttaaacaggaagagtatgattgaacaagatggattgc

SEQ ID NO: 33 - DNA, 50 bp, artificial
  Primer Pme-Kan-rev
aaaaaaccgtttaaacttggtctgacagtcagaagaactcgtcaagaagg

SEQ ID NO: 34 - DNA, 41 bp, artificial
  Primer Z-RBD-A85-forl
ctgccttatgaaannkctcaaggtgaggggcctgcaaccag

SEQ ID NO: 35 - DNA, 41 bp, artificial
  Primer Z-RBD-A85-rev
ccctcaccttgagmnntttcataaggcagtcatgcaagctc